(19) United States
(12) Patent Application Publication
(10) Pub. No.: US 2018/0360949 A1
(43) Pub. Date: Dec. 20, 2018

(54) IMMUNOGENIC COMPOSITIONS

(71) Applicant: GLAXOSMITHKLINE BIOLOGICALS, SA, Rixensart (BE)

(72) Inventors: Susan BARNETT, Rockville, MD (US); Marguerite Christine KOUTSOUKOS, Rixensart (BE); Clarisse Marie-Madeleine LORIN, Rixensart (BE); Frederick PORTER, Rockville, MD (US); Zihao WANG, Rockville, MD (US); Ying ZHANG, Rockville, MD (US)

(73) Assignee: GLAXOSMITHKLINE BIOLOGICALS, SA, Rixensart (BE)

(21) Appl. No.: 16/061,729

(22) PCT Filed: Dec. 16, 2016
(86) PCT No.: PCT/EP2016/081619

§ 371 Date: Jun. 13, 2018

Related U.S. Application Data
(60) Provisional application No. 62/268,715, filed on Dec. 17, 2015.

(51) Int. Cl. A61K 39/21; A61K 39/295; C07K 14/16

(52) U.S. Cl. CPC A61K 39/21; A61K 2039/55555; C07K 14/162; A61K 39/295

(57) ABSTRACT

The invention provides, inter alia, compositions useful for, e.g., raising an immune response to HIV-1, and associated methods of raising an immune response to HIV in a mammalian subject. In some embodiments, the compositions are bivalent immunogenic compositions comprising two (or, in some embodiments more than two) human immunodeficiency virus (HIV) clade C envelope gp120 polypeptide antigens, together with a liposome-based adjuvant, such as the adjuvant known as AS01.

### GOVERNMENT SUPPORT

This invention was made with government support under Contract No. HHSN272201300033C awarded by the National Institute of Allergy and Infectious Diseases, National Institutes of Health, Department of Health and Human Services. The government has certain rights in the invention.

### FIELD OF THE INVENTION

The invention relates to compositions useful for, for example, vaccines and methods of raising an immune response to human immunodeficiency virus (HIV).

### BACKGROUND OF THE INVENTION

HIV-1 remains a scourge to global health, imposing tremendous burdens on patients and their support networks, as well as local, national, and global relief agencies. Gp120 is the viral envelope glycoprotein expressed on the viral and infected cell surface(s) exposed to the humoral immune system and also the target for binding by many neutralizing and other functionally relevant antibodies.

Despite three decades of effort, a need remains for vaccine compositions useful for, e.g., raising protective immune responses to HIV-1.

### SUMMARY OF THE INVENTION

The invention provides, interalia, compositions useful for, e.g., raising an immune response to HIV-1, and associated methods of raising an immune response to HIV in a mammalian subject. The compositions are bivalent immunogenic compositions comprising two (or, in some embodiments more than two) human immunodeficiency virus (HIV) envelope gp120 polypeptide antigens, such as clade C gp120 polypeptide antigens, together with a liposome-based adjuvant, such as the adjuvant known as AS01. These compositions, when administered to a mammalian subject, such as a human, in an effective amount, elicit an immune response to HIV, e.g., an immune response to an HIV clade C envelope gp120 polypeptide antigen. Accordingly, these compositions can be formulated as pharmaceutical compositions and may be used in methods of raising an immune response to HIV in a mammalian subject, e.g., by administration in an effective amount to the subject.

### BRIEF DESCRIPTION OF THE FIGURES

FIG. 1 is a photographs of SDS-PAGE profiles of HIV TV1.C and 1086.C gp120. Reduced SDS-PAGE profile of TV1.C and 1086.C gp 120 before (lanes 1 and 2) and after (lane 3 and 4) de-N-glycosylation by PNGase F.

FIGS. 2A-2E are graphical depictions of antibody and CD4⁺ T cell responses induced by bivalent Clade C gp120 (1086.C and TV1.C) with or without Aluminium hydroxide or AS01 in CB6F1 mice. Animals were immunized intramuscularly on day 0, 14 and 28 with 2 μg of each gp120 (1086.C & TV1.C) alone or formulated with 50 μg of Al(OH)3 or 50 μl AS01. (A) anti-1086.C and (B) anti-TV1.C IgG binding antibody titers measured by ELISA 14 days post-second (blue) and third (red) immunization. (C) Anti-gp70-V1V2 (Clade B/Case A2) binding antibodies measured by ELISA at 14 days post-third dose. Each dot corresponds to individual animals. Statistical analysis: Analysis of variance (ANOVA), with multiplicity adjustment using the Tukey method. (D) 1086.C- and (E) TV1.C-specific CD4⁺ T cells secreting IFN-γ and/or IL-2 and/or TNFα were measured at 14 days post-third immunization. Intracellular staining was performed on splenocytes after a 6-hour re-stimulation with 1086 and TV1 Clade C gp120 antigens. 5 individual animals with medians are represented.

FIGS. 3A and 3B summarize characterization studies of N-linked glycosylation. (A) Sites and types of N-linked glycosylations in TV1.C (left panel) and 1086.C (right panel) gp120. In the left TV1.C panel: complex glycan at positions 59, 101, 110, 134, 168, 437 and 440; high mannose/hybrid glycan at positions 242, 256, 269, 275, 281, 304, 311, 318; glycan type undetermined at positions 106, 116, 119, 122, 138, 210, 214, 221, 364, 370 and 377; Complex/high mannose/hybrid at positions 177, 385, 418 and 424; non-glycosylated at position 334. In the right 1086.C panel: complex glycan at positions 55, 145. 356, 418 and 421 high mannose/hybrid glycan at positions 191, 195, 250, 294, 299, 345, 351 and 404; glycan type undetermined at positions 97, 104, 114, 202 and 360; Complex/high mannose/hybrid at positions 158, 237, 262, and 367. In both panels: Solid underscore: complete glycosylation; Dotted underscore - - - : incomplete glycosylation; N residues surrounded by a box: modified by a single HexNAc. (B) N-linked glycosylation profiling of gp120s. Top panel, TV1.C gp120; lower panel, 1086.C gp120.

FIG. 4 summarizes characterization studies of disulfide bonding patterns in TV1.C and 1086.C gp120s. Diagrams showing the overall disulfide bonding patterns in TV1.C (left panel) and 1086.C (right panel) gp120. Solid lines, expected disulfide bond linkages; dotted line, alternative disulfide bond linkages.

FIGS. 5A-5D are graphical summaries of studies on the Immunogenicity of bivalent Clade C gp120 (TV1.C and 1086.C)/AS01B in CB6F1 mice. Animals were intramuscularly immunized with 10 μg, 2 μg, 0.4 μg or 0.08 μg of bivalent (1086.C & TV1.C) gp120 antigen formulated in 50 μl of AS01_{B} at days 0, 14 and 28. (A,B) Percentage of 1086.C- and TV1.C-specific CD4+ T cells secreting IFN-γ and/or IL-2 and/or TNFα was measured at 7 days post-third immunization. Intracellular staining performed on PBLs after a 6-hour re-stimulation with 1086.C and TV1.C gp120 antigens. 4 pools of 6 mice with medians are represented. (C) Anti-TV1 and -1086 binding antibody titers measured by ELISA 14 days post-second and third immunization. (D) Anti-gp70-V1V2 (Clade B/Case A2) binding Ab titers. Each dot corresponds to individual animals for antibodies (Blue dots=14dpII; red dots=14dpIII).

FIG. 6 is a graphical summary of studies on the immunogenicity of bivalent Clade C gp120 (TV1.C and 1086.C) formulated in AS01B or MF59 in CB6F1 mice. Animals were intramuscularly immunized with 10 μg, 2 μg, or 0.4 μg of bivalent (1086.C & TV1.C) gp120 antigens (Tox lots 1023719 and 1023444) formulated in 50 μl of AS01B or 50 μl MF59 at days 0, 14 and 28. An additional group of mice was immunized with 2 μg of the gp120 consolidation lots (1086.C CR02 and TV1.C CR04) formulated in 50 μl AS01B as a control. Percentage of 1086.C- (A) and TV1.C- (B) specific CD4+ T cells secreting IFN-γ and/or IL-2 and/or TNFα was measured at 7 days post-third immunization. Intracellular staining performed on PBLs after a 6-hour re-stimulation with 1086.C and TV1.C gp120 antigens. 5 pools of 7 mice with medians are represented. Anti-1086.C (C) and TV1.C (D) binding antibody titers measured by ELISA 14 days post-third immunization. Each dot corresponds to individual animals for antibodies. Statistical analysis: Analysis of variance (ANOVA), with multiplicity adjustment using the Tukey method.

FIG. 7 shows anti-V1V2 Subtype C antibody responses induced by the bivalent Subtype C gp120 (1086.C and TV1.C) formulated with MF59 or AS01_{B} in CB6F1 mice. The animals were intramuscularly immunized with 10 μg, 2 μg, or 0.4 μg of bivalent (1086.C & TV1.C) gp120 antigens (Tox lots 1023719 and 1023444) formulated in 50 μl of AS01_{B} or 50 μl MF59 at days 0, 14 and 28. Anti-gp70-V1V2 Subtype C 1086.C (A) and TV1 (B) binding antibodies were measured by ELISA at 14 days and 77 days post-third dose. Geometric mean titers with 95% confidence interval are represented.

### DETAILED DESCRIPTION OF THE INVENTION

In one aspect, the invention provides compositions comprising two or more (e.g., 2, 3, 4, 5, 6, or more) different human immunodeficiency virus (HIV) clade C envelope gp120 polypeptide antigens and a liposome-based adjuvant. A “human immunodeficiency virus (HIV) clade C envelope gp120 polypeptide” or “HIV clade C envelope gp120 polypeptide” is an HIV group M, subgroup C gp120 protein, which is displayed on the surface of HIV. In other embodiments, other group M gp120 polypeptides can be used, such as clade A, B, D, E, F, G, H, I, J, K, or a combination thereof, including circulating recombinant forms (CRF) of any gp120 polypeptide, including CRFs of any of the foregoing, including clade C. In certain embodiments, a gp120 polypeptide antigen for use in the present invention retains the CD4 binding epitope, V1V2 loop integrity, V3 integrity, or a combination thereof, e.g., 1, 2 (any two), or all three.

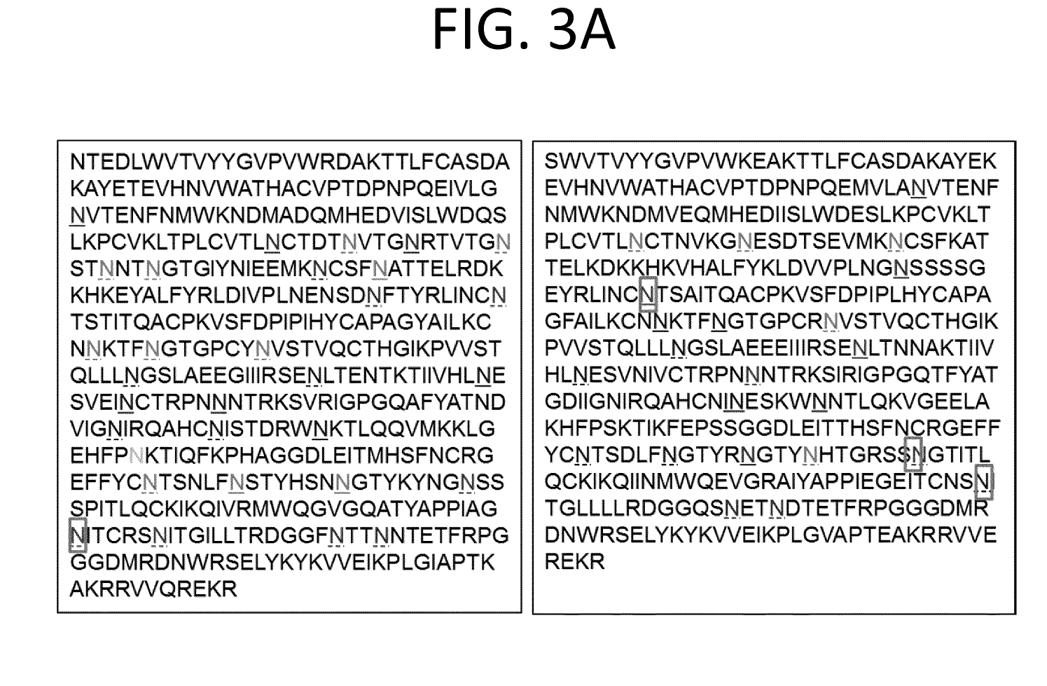

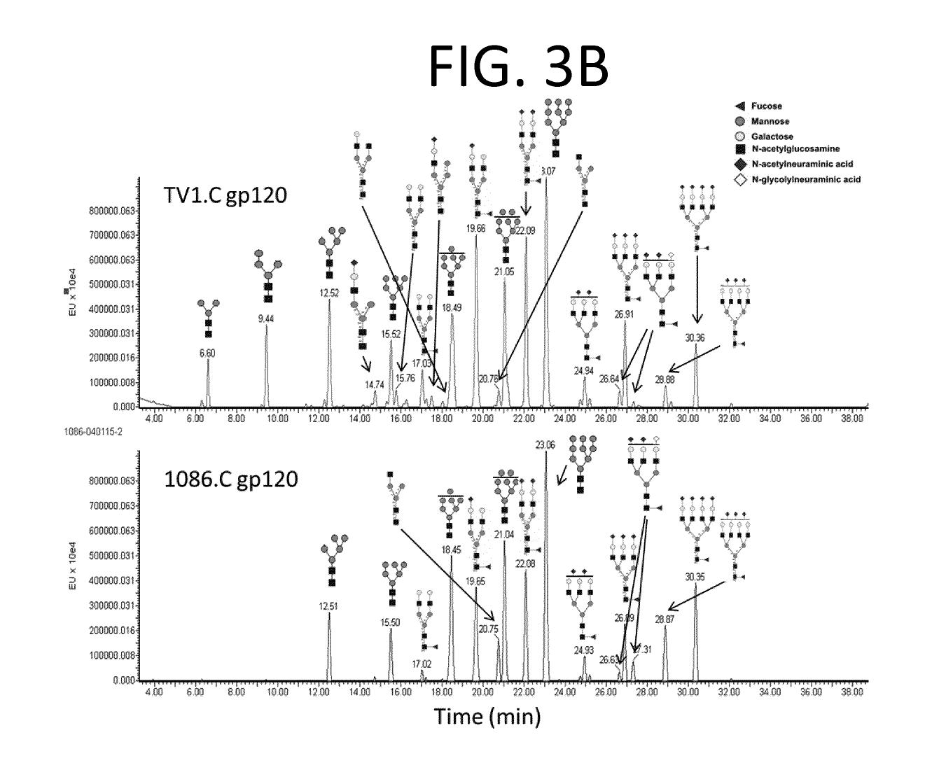

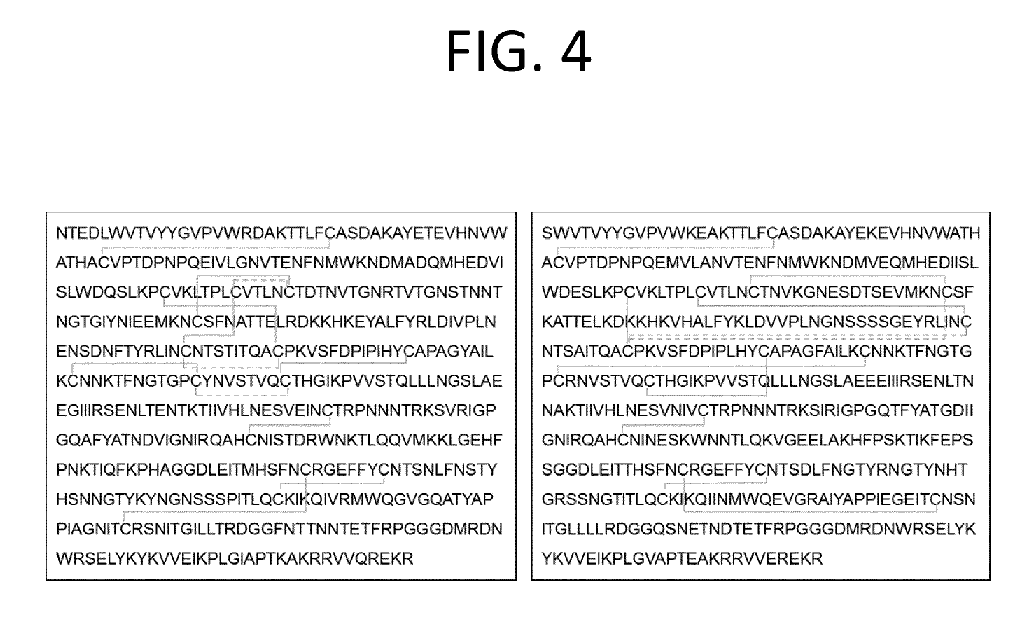

In certain embodiments, the HIV gp120 polypeptide, such as the HIV clade C envelope gp120 polypeptide, is glycosylated, comprising one or more O-linked, N-linked, or O-linked and N-linked glycan structures. In more particular embodiments, an HIV clade C envelope gp120 polypeptide exhibits a glycosylation pattern substantially as shown in FIG. 3, e.g., FIG. 3A. In certain embodiments, an HIV clade C envelope gp120 polypeptide exhibits a disulfide pattern substantially as shown in FIG. 4. In certain embodiments, one or more HIV clade C envelope gp120 polypeptide antigens for use in the invention may exhibit binding to PG09 (specific to the N159/60 glycosylated site) or PGT128 antibodies (specific to N332 glycosylated site).

A “liposome-based adjuvant” is an immuostimulatory compound comprising liposomes-lipid-based molecules comprising a lipid bilayer with one (unilamellar) or more (multilamellar) aqueous compartments within the bilayer. In some embodiments, a liposome-based adjuvant comprise immunostimulatory compounds in the lipid bilayer, such as a monophosphoryl lipid A or saponin. Liposome-based adjuvants are described in additional detail, below.

In a particular embodiment, the compositions provided by the invention comprise two HIV clade C envelope gp120 polypeptide antigens, optionally wherein the two polypeptide antigens are less than 95% identical to each other, such as less than 95, 94, 93, 92, 91, 90, 89, 88, 87, 86, 85, 84, 83, 82, 81, 80, 79, or 78% identical to each other; e.g., about 77.8% identical. Similarly, non-clade C gp120 polypeptide antigens may exhibit comparable degrees of sequence divergence, or even greater divergence, e.g., less than 80, 75, 70, or 65% identity.

In some embodiments, the one or more HIV clade C envelope gp120 polypeptide antigens in a composition provided by the invention exhibit a glycosylation pattern substantially as shown in FIG. 3A, a disulfide pattern substantially as shown in FIG. 4, or a glycosylation pattern substantially as shown in FIG. 3A and a disulfide pattern substantially as shown in FIG. 4.

In certain embodiments of the compositions of any one of the preceding embodiments, the HIV clade C envelope gp120 polypeptide antigens comprise, consist essentially of, or consist of an amino acid sequence having at least: 70, 75, 80, 85, 90, 91, 92, 93, 94, 95, 96, 97, 98, 99%, or more, identity to a sequence selected from SEQ ID NO:1, SEQ ID NO:2, or SEQ ID NO:1 and 2, such as at least 95, 96, 97, 98, 99, 99.1, 99.2, 99.3, 99.4, 99.5, 99.6, 99.7, 99.8, 99.9% identity to SEQ ID NO:1, SEQ ID NO:2, or SEQ ID NO:1 and 2.

Gp120 polypeptide antigens for use consonant with the invention, such as HIV clade C envelope gp120 polypeptide antigens, can be made by any suitable means. In some embodiments, the dose of polypeptide in a compositions provided by the invention, i.e., the human unit dose per immunization, per antigen, is between about 10 μg and about 400 μg, e.g., about: 10, 20, 30, 40, 50, 60, 70, 80, 90, 100, 110, 120, 130, 140, 160, 180, 200, 225, 250, 275, 300, 325, 350, 375, 400 μg, or more. In particular embodiments, the human unit dose may be about 20 μg (e.g., 10-30 μg or 15-25 μg) or about 100 μg (e.g., 75-125 μg or 90-110 μg). Accordingly, for example, in embodiments where two HIV clade C envelope gp120 polypeptide antigens are employed, the total amount of HIV clade C envelope gp120 polypeptide antigens will be double the per-antigen dose (i.e. there is double the amount of antigen when two gp120 polypeptide antigens are used). Analogously, for three HIV clade C envelope gp120 polypeptide antigens, the total amount of antigens will be triple the above per-antigen dose (i.e. there is three times the amount of antigen when three gp120 polypeptide antigens are used), et cetera.

In some embodiments of a composition of any one of the preceding embodiments, the liposome-based adjuvant comprises a phosphatidylcholine (PC) and a sterol. In certain particular embodiments, the PC is 1,2-dioleoyl-sn-glycero-3-phosphocholine (DOPC), the sterol is cholesterol, or the PC is DOPC and the sterol is cholesterol.

In certain embodiments of any of the preceding embodiments, the composition further comprises a lipophilic or amphipathic immunostimulant. In more particular embodiments, the immunostimulant is selected from a monophosphoryl lipid A (MPL), a saponin or an MPL and a saponin. In certain embodiments, the saponin is derived from Quillaja saponaria, such as Quillaja saponaria bark, such as QS-21 (Quillaja saponaria Molina, fraction 21). In some particular embodiments the MPL is 3-O-desacyl-4′-monophosphoryl lipid A.

In some particular embodiments, a composition of any one of the preceding embodiments, further comprises a pharmaceutically acceptable excipient. In certain embodiments, suitable excipients include buffers and tonicity agents; in more particular embodiments, the excipients include sodium citrate, citric acid, sodium chloride, sodium phosphate, potassium phosphate, and combinations of the foregoing.

In certain embodiments the composition of any one of the preceding embodiments is in lyophilized form. In other embodiments, the composition of any one of the preceding embodiments is in aqueous form. In any of the preceding embodiments, the composition may be in unit dosage form.

In some embodiments, the composition of any one of the preceding embodiments is for use in medicine, for example, for use in the treatment or prevention of HIV.

In some embodiments, the composition of any one of the preceding embodiments, when administered in an effective amount to a mammalian subject, elicits an immune response to HIV, optionally wherein the immune response is at least a partially protective immune response. A partially protective immune response may be, on a population level, at least about: 10, 15, 20, 25, 30, 35, 40, 45, 50, 55, or 60% protective (against HIV acquisition) at about: 3, 6, 9, 12, 15, 18, 24, 27, 30, 33, 36 months, or more. In some embodiments, a partially protective immune response means the P5 TPP (target product profile) is greater than or equal to about 50% protection against infection with onset after second boost at about 6 months and with a duration of 36 months. In certain embodiments, the immune response is a minimum target efficacy profile for a preventive HIV vaccine, e.g., protection against HIV infection for a duration of at least about 2 years after first administration, optionally with one or more boosts. Accordingly, in a related aspect, the composition of any one of the preceding embodiments can be for use in raising an immune response to HIV in a mammalian subject, optionally wherein the mammalian subject is a human, more particularly wherein the human is an adult or adolescent, such as an adolescent prior to sexual debut, a pediatric subject, or neonate, such as a neonate born of an HIV+ mother.

In some embodiments, a higher anti-V1V2 antibody response is obtained for a composition comprising the liposome-based adjuvant (for example, a liposome-based adjuvant comprising QS-21 and 3-O-desacyl-4′-monophosphoryl lipid A) than for an otherwise identical composition having MF59 as the adjuvant.

The invention also provides a method of vaccinating a mammalian subject optionally wherein the mammalian subject is a human, using the composition of any one of the preceding embodiments.

In a related aspect, the invention provides uses of the compositions of any of the proceeding aspects and embodiments in the manufacture of a medicament for raising an immune response to HIV in a mammalian subject, optionally wherein the mammalian subject is a human, more particularly wherein the human is an adult.

In a related aspect, the invention provides methods of raising an immune response to HIV in a mammalian subject. These methods include a step of administering an effective amount of the composition of any one of the preceding aspects or embodiments to a subject, e.g., wherein the mammalian subject is a human, more particularly wherein the human is an adult.

In some embodiments for any of the preceding aspects or embodiments, a subject treated, or to be treated, by a method provided by the invention, or administered a composition of the invention, is a human that has previously been (or, in other embodiments, is concurrently or sequentially)—one or more times—administered a nucleic acid (e.g., DNA or RNA) encoding one or more HIV antigens. In certain embodiments, the nucleic acid encodes HIV env, gag, pol, or a combination thereof. In more particular embodiments, the nucleic acid is in the form of a viral vector, such as an inert canarypox vector, or an MVA or NYVAC pox vector. In other embodiments, the viral vector may be an adenovirus vector, such as human or chimp adenovirus vector. In some embodiments, a “priming” administration of a nucleic acid-containing composition can be concurrent or sequential with administering a composition provided by the invention. For example, in some embodiments, the nucleic acid containing composition is administered before (e.g., 1, 2, 3, 4, 5, 6, or 7 days, 1, 2, 3, 4, 5, 6, or more weeks, or 1, 2, 3, 4, 5, or 6 months) a composition comprising an HIV clade C envelope gp120 polypeptide provided by the invention. In other embodiments, a nucleic acid composition is administered substantially concurrently with a composition provided by the invention. In certain embodiments, a nucleic acid-containing composition was previously administered to a subject—one or more times—and then may be administered substantially concurrently—one or more times—with a HIV clade C envelope gp120 polypeptide-containing composition provided by the invention. For example, in particular embodiments, substantially concurrent administration of nucleic acid and polypeptide-containing compositions may take place in opposite deltoids, or other large muscles, e.g., with nucleic acid in the right (or left) and polypeptide in the left (or right). In other embodiments, a subject is not, or has not been, administered a nucleic acid encoding one or more HIV antigens.

Suitably, the polypeptides used in the present invention are isolated. An “isolated” polypeptide is one that is removed from its original environment. For example, a naturally-occurring protein is isolated if it is separated from some or all of the coexisting materials in the natural system. Preferably, such polypeptides are at least about 90% pure, more suitably at least about 95% pure.

Polypeptides used consonant with the invention can be produced by any acceptable means, such as complete synthesis, or recombinant expression in a suitable cell line, such as insect cells, CHO cells, COS cells, 293 cells, or PerC6 cells.

### Adjuvant

Saponins

The immunogenic composition of the invention comprises an immunologically active saponin fraction (“a saponin”) as an adjuvant or as a component of an adjuvant. A particularly suitable saponin for use in the present invention is Quil A and its derivatives. Quil A is a saponin preparation isolated from the South American tree Quillaja Saponaria Molina and was first described by Dalsgaard et al. in 1974 (“Saponin adjuvants”, Archiv. für die gesamte Virusforschung, Vol. 44, Springer Verlag, Berlin, p 243-254) to have adjuvant activity. Purified fragments of Quil A have been isolated by HPLC which retain adjuvant activity without the toxicity associated with Quil A (U.S. Pat. No. 5,604,106), for example QS-7 and QS-21 (also known as QA7 and QA21). QS-21 is a natural saponin derived from the bark of Quillaja saponaria Molina, which induces CD8+ cytotoxic T cells (CTLs), Th1 cells and a predominant IgG2a antibody response in mice and is a preferred saponin in the context of the present invention.

In a suitable form of the present invention, the saponin adjuvant within the immunogenic composition is a derivative of saponaria molina quil A, suitably an immunologically active fraction of Quil A, such as QS-7 or QS-21, suitably QS-21. In one embodiment the compositions of the invention contain the immunologically active saponin fraction in substantially pure form. Suitably the compositions of the invention contain QS-21 in substantially pure form, that is to say, the QS-21 is at least 90% pure, for example at least 95% pure, or at least 98% pure.

In a specific embodiment, QS-21 is provided in a less reactogenic composition where its lytic activity is quenched with an exogenous sterol, such as cholesterol for example. Several particular forms of less reactogenic compositions wherein the lytic activity of QS-21 is quenched with an exogenous cholesterol exist. In a specific embodiment, the saponin/sterol is in the form of a liposome structure (U.S. Pat. No. 6,846,489, Example 1). In this embodiment the liposomes suitably contain a neutral lipid, for example phosphatidylcholine, which is suitably non-crystalline at room temperature, for example egg yolk phosphatidylcholine, dioleoyl phosphatidylcholine (DOPC) or dilauryl phosphatidylcholine. The liposomes may also contain a charged lipid which increases the stability of the lipsome-QS-21 structure for liposomes composed of saturated lipids. In these cases the amount of charged lipid is suitably 1-20% w/w, suitably 5-10%. The ratio of sterol to phospholipid is 1-50% (mol/mol), suitably 20-25%.

Suitable sterols include beta-sitosterol, stigmasterol, ergosterol, ergocalciferol and cholesterol. In one particular embodiment, the adjuvant composition comprises cholesterol as sterol. These sterols are well known in the art, for example cholesterol is disclosed in the Merck Index, 11th Edn., page 341, as a naturally occurring sterol found in animal fat.

The sterol according to the invention is taken to mean an exogenous sterol, i.e. a sterol which is not endogenous to the organism from which the antigenic preparation is taken but is added to the antigen preparation or subsequently at the moment of formulation. Typically, the sterol may be added during subsequent formulation of the antigen preparation with the saponin adjuvant, by using, for example, the saponin in its form wherein its lytic activity is quenched with the sterol. Suitably the exogenous sterol is associated to the saponin adjuvant as described in U.S. Pat. No. 6,846,489.

Where the active saponin fraction is QS-21, the ratio of QS-21: sterol will typically be in the order of 1:100 to 1:1 (w/w), suitably between 1:10 to 1:1 (w/w), and suitably 1:5 to 1:1 (w/w). Suitably excess sterol is present, the ratio of QS-21: sterol being at least 1:2 (w/w). In one embodiment, the ratio of QS-21: sterol is 1:5 (w/w).

Other saponins which have been described in the literature include Escin, which has been described in the Merck index (12th ed: entry 3737) as a mixture of saponins occurring in the seed of the horse chestnut tree, Lat: Aesculus hippocastanum. Its isolation is described by chromatography and purification (Fiedler, Arzneimittel-Forsch. 4, 213 (1953)), and by ion-exchange resins (Erbring et al., U.S. Pat. No. 3,238,190). Fractions of escin have been purified and shown to be biologically active (Yoshikawa M, et al. (Chem Pharm Bull (Tokyo) 1996 44(8):1454-1464)). Sapoalbin from Gypsophilla struthium (R. Vochten et al., 1968, J Pharm Belg, 42, 213-226) has also been described in relation to ISCOM production for example. Another useful saponin is those derived from the plant Gyophilla struthium.

Suitably, the total amount of saponin in the immunogenic composition of the present invention, particularly in a human dose of the immunogenic composition of the present invention is between 1-100 ug.

In one embodiment, there is provided an immunogenic composition comprising QS-21 at a level of around 50 μg, for example between 38-100 μg, suitably between 40-75 μg or between 45-60 μg, more suitably 49-51, most suitably 50 μg.

In a further embodiment, there is provided an immunogenic composition comprising QS-21 at a level of around 25 μg, for example between 10-37 μg, suitably between 15-30 μg or between 20-27 μg, more suitably 24-26, more suitably 25 μg.

In another embodiment, there is provided an immunogenic composition in a volume which is suitable for a human dose which human dose of the immunogenic composition comprises QS-21 at a level of around 50 μg, for example between 38-100 μg, suitably between 40-75 μg or between 45-60 μg, more suitably 49-51, most suitably 50 μg.

In another embodiment, there is provided an immunogenic composition in a volume which is suitable for a human dose which human dose of the immunogenic composition comprises QS-21 at a level of around 25 μg, for example between 10-37 μg, suitably between 15-30 μg or between 20-27 μg, more suitably 24-26, more suitably 25 μg.

The dose of QS-21 is suitably able to enhance an immune response to an antigen in a human. In particular a suitable QS-21 amount is that which improves the immunological potential of the composition compared to the unadjuvanted composition, or compared to the composition adjuvanted with another QS-21 amount, whilst being acceptable from a reactogenicity profile.

Lipopolysaccharide Adjuvants

Lipopolysaccharides (LPS) are the major surface molecule of, and occur exclusively in, the external leaflet of the outer membrane of gram-negative bacteria. LPS impede destruction of bacteria by serum complements and phagocytic cells, and are involved in adherence for colonisation. LPS are a group of structurally related complex molecules of approximately 10,000 Daltons in size and consist of three covalently linked regions:
(i) an O-specific polysaccharide chain (O-antigen) at the outer region
(ii) a core oligosaccharide central region
(iii) lipid A—the innermost region which serves as the hydrophobic anchor, it comprises glucosamine disaccharide units which carry long chain fatty acids.

The biological activities of LPS, such as lethal toxicity, pyrogenicity and adjuvanticity, have been shown to be related to the lipid A moiety. In contrast, immunogenicity is associated with the O-specific polysaccharide component (O-antigen). Both LPS and lipid A have long been known for their strong adjuvant effects, but the high toxicity of these molecules has precluded their use in vaccine formulations. Significant effort has therefore been made towards reducing the toxicity of LPS or lipid A while maintaining their adjuvanticity.

The Salmonella minnesota mutant R595 was isolated in 1966 from a culture of the parent (smooth) strain (Luderitz et al. 1966 Ann N Y Acad Sci 133:349-374). The colonies selected were screened for their susceptibility to lysis by a panel of phages, and only those colonies that displayed a narrow range of sensitivity (susceptible to one or two phages only) were selected for further study. This effort led to the isolation of a deep rough mutant strain which is defective in LPS biosynthesis and referred to as S. minnesota R595.

In comparison to other LPS, those produced by the mutant S. minnesota R595 have a relatively simple structure:

(i) they contain no O-specific region—a characteristic which is responsible for the shift from the wild type smooth phenotype to the mutant rough phenotype and results in a loss of virulence

(ii) the core region is very short—this characteristic increases the strain susceptibility to a variety of chemicals

(iv) the lipid A moiety is highly acylated with up to 7 fatty acids.

4′-monophosporyl lipid A (MPL), which may be obtained by the acid hydrolysis of LPS extracted from a deep rough mutant strain of gram-negative bacteria, retains the adjuvant properties of LPS while demonstrating a toxicity which is reduced by a factor of more than 1000 (as measured by lethal dose in chick embryo eggs) (Johnson et al. 1987 Rev Infect Dis 9 Suppl:S512-S516). LPS is typically refluxed in mineral acid solutions of moderate strength (e.g. 0.1 M HCl) for a period of approximately 30 minutes. This process results in dephosphorylation at the 1 position, and decarbohydration at the 6′ position, yielding MPL.

3-O-deacylated monophosphoryl lipid A (3D-MPL), which may be obtained by mild alkaline hydrolysis of MPL, has a further reduced toxicity while again maintaining adjuvanticity, see U.S. Pat. No. 4,912,094 (Ribi Immunochemicals). Alkaline hydrolysis is typically performed in organic solvent, such as a mixture of chloroform/methanol, by saturation with an aqueous solution of weak base, such as 0.5 M sodium carbonate at pH 10.5. Further information on the preparation of 3D-MPL is available in, for example, U.S. Pat. No. 4,912,094 (Corixa Corporation).

The composition may further comprise an additional adjuvant which is a lipopolysaccharide, suitably a non-toxic derivative of lipid A, particularly monophosphoryl lipid A or more particularly 3-deacylated monophoshoryl lipid A (3D-MPL).

3D-MPL is sold under the name MPL by GlaxoSmithKline Biologicals N.A. and is referred throughout the document as MPL or 3D-MPL. see, for example, U.S. Pat. No. 4,436,727; U.S. Pat. No. 4,877,611; U.S. Pat. No. 4,866,034 and U.S. Pat. No. 4,912,094. 3D-MPL can be produced according to the methods disclosed in U.S. Pat. No. 4,912,094. Chemically it is a mixture of 3-deacylated monophosphoryl lipid A with 3, 4, 5 or 6 acylated chains. Suitably in the compositions of the present invention small particle 3D-MPL is used. Small particle 3D-MPL has a particle size such that it may be sterile-filtered through a 0.22 um filter. Such preparations are described in U.S. Pat. No. 5,776,468.

Suitably, the total amount of lipopolysaccharide in the immunogenic composition of the present invention, particularly in a human dose of the immunogenic composition of the present invention, is between 1-100 ug.

In one embodiment, there is provided an immunogenic composition comprising 3D-MPL at a level of around 50 μg, for example between 38-100 μg, suitably between 40-75 μg or between 45-60 μg, more suitably 49-51, most suitably 50 μg.

In a further embodiment, there is provided an immunogenic composition comprising 3D-MPL at a level of around 25 μg, for example between 10-37 μg, suitably between 15-30 μg or between 20-27 μg, more suitably 24-26, more suitably 25 μg.

In another embodiment, there is provided an immunogenic composition in a volume which is suitable for a human dose which human dose of the immunogenic composition comprises 3D-MPL at a level of around 50 μg, for example between 38-100 μg, suitably between 40-75 μg or between 45-60 μg, more suitably 49-51, most suitably 50 μg.

In another embodiment, there is provided an immunogenic composition in a volume which is suitable for a human dose which human dose of the immunogenic composition comprises 3D-MPL at a level of around 25 μg, for example between 10-37 μg, suitably between 15-30 μg or between 20-27 μg, more suitably 24-26, more suitably 25 μg.

Suitable compositions of the invention are those wherein liposomes are initially prepared without MPL (as described in U.S. Pat. No. 6,846,489), and MPL is then added, suitably as small particles of below 100 nm particles or particles that are susceptible to sterile filtration through a 0.22 um membrane. The MPL is therefore not contained within the vesicle membrane (known as MPL out). Compositions where the MPL is contained within the vesicle membrane (known as MPL in) also form an aspect of the invention. The antigen can be contained within the vesicle membrane or contained outside the vesicle membrane. Suitably soluble antigens are outside and hydrophobic or lipidated antigens are either contained inside or outside the membrane.

The invention may comprise both lipopolysaccharide and immunologically active saponin. In a specific embodiment of the invention, the lipopolysaccharide is 3D-MPL and the immunologically active saponin is QS-21. In an embodiment of the invention, the composition comprises a lipopolysaccharide and immunologically active saponin in a liposomal formulation. Suitably in one form of these embodiments, the composition comprises 3D-MPL and QS-21, with optionally a sterol which is suitably cholesterol.

In a further embodiment of the invention, the adjuvant composition comprises in a liposomal formulation lipopolysaccharide and immunologically active saponin in combination with one or more further immunostimulants or adjuvants. Suitably in one form of this embodiment the lipopolysaccharide is 3D-MPL and the immunologically active saponin is QS-21.

In a specific embodiment, QS-21 and 3D-MPL are present in a weight ratio of between 1:2 and 2:1. Suitably QS-21 and 3D-MPL are present in same final amount per human dose of the immunogenic composition. In one aspect of this embodiment, a human dose of immunogenic composition comprises a final level of about 50 μg of 3D-MPL and about 50 μg of QS-21. In another aspect, a human dose of immunogenic composition comprises a final level of about 25 μg of 3D-MPL and about 25 μg of QS-21. In a further embodiment, a human dose of immunogenic composition comprises a final level of about 10 ug each of MPL and QS-21.

Vaccine preparation is generally described in New Trends and Developments in Vaccines, edited by Voller et al., University Park Press, Baltimore, Md., U.S.A. 1978. Encapsulation within liposomes is described, for example, in U.S. Pat. No. 4,235,877. Conjugation of proteins to macromolecules is disclosed, for example, in U.S. Pat. No. 4,372,945 and in U.S. Pat. No. 4,474,757.

### Immunogenic Properties of the Immunogenic Composition of the Present Invention

In the present invention the immunogenic composition is suitably capable of inducing humoral response in a mammal, such as a human, administered with the immunogenic composition.

Humoral responses can be detected using an appropriate antibody-based assay. For example, the presence in serum of an immunoglobulin G (IgG) antibody response to a HIV clade C envelope gp120 polypeptide antigen can be analyzed using ELISA. The induction of potent humoral responses such as IgG antibodies, including IgG antibodies binding the gp120 protein or a region of the gp120 such as the variable loop structures such as the V1V2 of gp120, indicates the immunogenicity of the immunogenic compositions of the invention. In some embodiments, the IgG response is IgG1, IgG3, or IgG1 and IgG3. In certain embodiments, the compositions provided by the invention elicit polyfunctional antibody responses, including virus neutralizing responses and other functional antibody responses such as ADCC, ADCP, ADCVI, CD107a degranulation, IFN-gamma, MIP-1 beta, polyfunctional NK activation, virion capture, virus neutralization, C mediated effector function and combinations thereof, including 2, 3, 4, 5, 6, 7, 8, or all 9 of the foregoing. In some embodiments, the compositions provided by the invention are effective to raise anti-V3 responses and response to other HIV gp120 specific epitopes. Additional efficacious properties and assays to detect them are known in the art and are described in, for example, Garcia-Arriaza et al., J. Virol 89(16):8525-39 (2015), which is incorporated by reference.

In a further embodiment, the immunogenic composition is capable of inducing an improved CD4+ T-cell immune response.

By “improved CD4 T-cell immune response” is meant that a higher CD4 response is obtained in a mammal, such as a human, after administration of the adjuvanted immunogenic composition as compared to a suitable control. In some embodiments, a suitable control is an earlier time point (e.g., before administration of a composition provided by the invention to the subject). In other embodiments, a suitable control is relative to a composition containing no gp120 polypeptide antigen, e.g., in a control group. In other embodiments, a suitable control is a composition comprising a gp120 polypeptide formulated with a liposomal adjuvant without QA-21 or MPL, or a non-liposomal adjuvant, such as alum or MF59, or a formulation without adjuvant.

In particular but not exclusively, said ‘improved CD4 T-cell immune response’ is obtained in an immunologically unprimed patient, i.e. a patient who is seronegative to HIV In other particular embodiments, the response may be achieved in a subject that is HIV+.

The improved CD4 T-cell immune response (which may be provided by ‘polyfunctional’ T cells) may be assessed by measuring the number of cells producing any of the following immune markers:
CD4 T cells that express at least one immune marker (e.g. IL-2, IL-4, CD40L, IFN-gamma, TNF-alpha, or in particular embodiments, a combination thereof, e.g., 2, 3, 4, or all 5 markers)
cells producing at least two different immune markers (e.g. CD40L, IL-2, IL-4, IFN-gamma, TNF-alpha, or in particular embodiments, a combination thereof, e.g., 2, 3, 4, or all 5 markers)
cells producing at least CD40L and another immune marker (e.g. IL-2, IL-4, TNF-alpha, IFN-gamma, or a combination thereof, e.g., 2, 3, 4, or all 5 markers• cells producing at least IL-2 and another immune marker (e.g., CD40L, IL-4, TNF-alpha, IFN-gamma, or a combination thereof, e.g., 2, 3, 4, or all 5 markers)
cells producing at least IFN-gamma and another immune marker (e.g. IL-2, IL-4, TNF-alpha, CD40L, or a combination thereof, e.g., 2, 3, 4, or all 5 markers)
cells producing at least TNF-alpha and another immune marker (e.g. IL-2, IL-4, CD40L, IFN-gamma, or a combination thereof, e.g., 2, 3, 4, or all 5 markers)

There will be an improved CD4 T-cell immune response when cells producing any of the above immune markers are in a higher amount following administration. Typically at least one, suitably two of the six conditions mentioned herein above will be fulfilled. In a particular embodiment, the cells producing all four immune markers will be present at a higher amount.

The improved CD4 T-cell immune response conferred by the HIV clade C envelope gp120 polypeptide antigen-containing composition of the present invention may be obtained after one single administration, or in other embodiments, after more than one administration, such as two or three administrations.

In another embodiment, the administration of said immunogenic composition induces an improved B-memory cell response in a mammal, such as a human, administered with the immunogenic composition. An improved B-memory cell response is intended to mean an increased frequency of peripheral blood B lymphocytes capable of differentiation into antibody-secreting plasma cells upon antigen encounter as measured by stimulation of in-vitro differentiation.

In a specific embodiment, the administration of said immunogenic composition induces at least two of the following responses: (i) an improved CD4 T-cell immune response, (ii) an improved B-memory cell response, (iii) an improved humoral response, against at least one of the component antigen(s) or antigenic composition compared to either immune response obtained with other compositions.

The magnitude of an immune response can also be expressed as the titre (or concentration) of antigen-specific antibodies induced by the immunogenic composition as determined by an appropriate serological test. The magnitude of a T cell response can be expressed as the frequency (or number) of antigen-specific cells induced by the immunogenic composition among the total population of T cells, which can be monitored by cytokine production.

In particular embodiments, immune responses elicited by the compositions and methods provided by the invention are polyfunctional, e.g., both polyfunctional antibody and cellular immune responses as described supra. Methods for detecting and assessing these responses are known in the art, e.g., Chung et al., Science Transl. Med., Vol 6, Iss. 228 228ra38 (2014), Yates et al., Science Transl. Med., Vol 6, Iss. 228 228ra39 (2014), and Lin et al., Nature Biotech. 33:610-18 (2015), which are incorporated by reference.

Suitably the composition of the present invention elicits an immune response capable of cross-reactivity. Cross-reactivity is herein taken to mean the ability of immune responses induced by an immunogenic composition of the invention to recognize strains of HIV-1 from subtypes that are not represented in the immunogenic composition. For example, an immunogenic composition of the invention comprising a gp120 related polypeptide comprising from a strain of HIV-1 from subtype C is considered cross-reactive if the HIV-specific immune response, such as HIV-specific antibody or CD4+ T cell response (in particular an antibody response to the V1V2 loop of gp120), induced by the composition reacted with one or more different strains of HIV-1 not in the composition, for example, with a strain of HIV-1 from a subtype other than subtype C. Suitably, cross-reactivity will be in respect of an HIV-1 strain from a different sub-type, in particular in respect of an HIV-1 strain from a different group. In some embodiments cross reactivity is between different subtypes in the same clade.

Suitably, the level of cross-reactivity observed is up to 10%, up to 15%, up to 20%, up to 25%, up to 30%, up to 35%, up to 40%, up to 45%, up to 50%, up to 55%, up to 60%, up to 65% up to 70%, up to 80%, up to 90% or up to 100% of antigen-specific cells induced by the immunogenic composition among the total population of T cells or titre (or concentration) of antigen-specific antibodies induced by the immunogenic composition.

When measuring cross-reactivity in terms of the percentage of responders to the strains of HIV-1 from different subtypes, the number or percentage of vaccinated individuals that show a positive response in an immunological assay after subsequent challenge can be measured. A responder can respond to one or more epitopes on an antigen. A responder can also respond to one or more polypeptides in an immunogenic composition of the invention and/or to one or more antigens in an immunogenic composition of the invention.

Immunological assays such as serological tests that can be used to analyse the percentage of responders or the magnitude of an immune response are known in the art. Examples of such assays are known to a person skilled in the art.

Suitably, the level of cross-reactivity observed is up to 10%, up to 15%, up to 20%, up to 25%, up to 30%, up to 35%, up to 40%, up to 45%, up to 50%, up to 55%, up to 60%, up to 65% up to 70%, up to 80%, up to 90% or up to 100% of subjects in a sample are responders.

In an embodiment, the immunogenic composition of the invention is for use in eliciting high and long-lasting numbers of HIV-1-specific antibodies in an individual not infected with HIV.

In a further embodiment, the immunogenic composition of the invention is for use in eliciting high and long-lasting numbers of HIV-1-specific antibodies in an individual at risk of infection with an HIV-1 strain from one or more clades different from the one or more HIV-1 clades from which the HIV clade C envelope gp120 polypeptide antigen in the immunogenic composition is derived.

In an embodiment, the immunogenic composition of the invention is for use in controlling or reducing viremia in an individual infected with HIV.

Suitably, after administration of the composition, the viral load of the subject remains below 100,000 copies/ml for at least four months after administration. In a further embodiment, the viral load of the subject remains below 100,000 copies/ml of serum for at least six months, at least twelve months, at least eighteen months, at least two years, at least three years, at least four years, at least five years, at least six years, at least seven years, at least eight years, at least nine years, or at least ten years. In another embodiment, the subject maintains a viral load below 50,000 copies/ml, below 10,000 copies/ml, below 5000 copies/ml, below 1000 copies/ml, or below 500 copies/ml. Suitably, viral load is maintained or reduced for at least six months, at least twelve months, at least eighteen months, at least two years, at least three years, at least four years, at least five years, at least six years, at least seven years, at least eight years, at least nine years, or at least ten years after administration of the composition.

Suitably, administration of the inventive composition results in a durable response. A durable response is for example the ability to detect, in the serum of an individual, IgG antibody capable of binding to the V1V2 region of the HIV clade C envelope gp120 polypeptide antigen of the composition at least 24 weeks, at least 48 weeks, at least 72 weeks, at least 96 weeks, at least two years, at least three years, at least four years, at least five years, at least six years, at least seven years, at least eight years, at least nine years, or at least ten years after the only administration of the composition, or the first administration of the composition in a course of repeat administrations, to the individual. Suitably, antibody levels will be detected at a level of at least 5%, more suitably at least 10% and in particular at least 20% of the serum titre two weeks following the first administration. Suitably the antibody will be detectable in at least 50% of individuals, more suitably at least 60% of individuals and in particular at least 75%.

Suitably, a durable response is for example the ability to detect, in the serum of an individual, IgG antibody binding the V1V2 region of an HIV envelope gp120 polypeptide antigen (e.g., from clade C, or another clade, or in some embodiments, clade C and another clade) of the composition at least 2 weeks, at least 6 months, at least 12 months, at least 18 months, at least two years, at least three years, at least four years, at least five years, at least six years, at least seven years, at least eight years, at least nine years, or at least ten years after the final administration of the composition in a course of repeat administrations to the individual. Suitably, antibody levels will be detected at a level of at least 5%, more suitably at least 10% and in particular at least 20% of the serum titre two weeks following the final administration. Suitably the antibody will be detectable in at least 50% of individuals, more suitably at least 60% of individuals and in particular at least 75%.

Suitably, the present invention is capable of achieving a more durable immune response based on responder rates. Suitably, up to 10%, up to 15%, up to 20%, up to 25%, up to 30%, up to 35%, up to 40%, up to 45%, up to 50%, up to 55%, up to 60%, up to 65% up to 70%, up to 80%, up to 90% or up to 100% of vaccinated individuals mount an increased humoral response such as an increased serum level of IgG antibody binding the V1V2 region of the gp120 related polypeptide of the composition.

Vaccination Means

The immunogenic compositions of the invention may be administered by any suitable delivery route, such as intradermal, mucosal e.g. intranasal, oral, intramuscular or subcutaneous. Other delivery routes are well known in the art. The intramuscular delivery route is preferred for certain embodiments of the immunogenic composition and methods using them.

Various delivery regimens, such as prime-boost with nucleic acid containing compositions and protein-containing compositions can be employed as already described supra. For example, priming with nucleic acids and boosting with polypeptide-containing compositions provided by the invention.

### EXAMPLES

### Example 1: Antigen Testing

### Introduction

Human immunodeficiency virus type 1 (HIV-1) entry is dependent on envelope glycoprotein (Env), which consists of two non-covalently bound subunits, the external glycoprotein gp120 and the transmembrane glycoprotein gp41. Env is the only protein on the viral surface exposed to humoral immune system and also the target for the binding of many neutralizing antibodies. Thus, it has been a natural choice for development of antibody-based vaccines against HIV-1. The RV144 clinical trial conducted in Thailand, which showed 31.2% efficacy 3.5 years after vaccination and potentially up to 60% within 1 year time frame, was the first trial that demonstrated a vaccine could protect against HIV infection. The RV144 experimental vaccine is a ‘prime-boost’ scheme consisting of a canarypox viral vector encoding HIV Env, Gag, and Pol proteins (ALVAC-HIV, prime) and a recombinant gp120 protein (AIDSVAX B/E, boost). Follow-up studies suggested that antibodies targeting gp120 V1/V2 loops were associated with the reduced infection risk. The next series of HIV vaccine proof-of-concept clinical trials planned for the southern African regions aim to confirm and extend the partial protection demonstrated by RV144 with altered antigen design, dose, and vaccination schedule. The ‘Pox Protein Public Private Partnership (P5)’ partnered to produce ˜50,000 doses each of two selected subtype C gp120 vaccine antigens, namely TV1.C and 1086.C, for use together with a proprietary adjuvant for the South African trials. Both TV1.C and 1086.C gp120s originate from HIV-1, Group M, Subtype C, which has high incidence of infection in South Africa. TV1. C is the chronic form, while 1086.C is the early transmitted/founder form of this virus subtype. Both gp120s were recombinantly expressed in Chinese Hamster Ovary (CHO) cells (CHO K1 for TV.1 C gp120 and CHO K1-PD for 1086.C gp120) as secreted glycoproteins and subsequently purified from culture media.

Over the past few decades, a number of highly potent broadly neutralizing antibodies (bNAbs) have been discovered, some of which could suppress HIV replication and entry into CD4⁺ cells. Viral epitopes of these bNAbs often involve molecular structural elements of gp120. For example, 2G12 recognizes the oligomannose clusters on gp120, most likely N295, N332, and N392; PGT128 recognizes two high mannose glycans (N301 and N332) and a short f-strand segment of V3 loop on gp120; PG9/16 epitope involves one high-mannose glycan (N160), one complex/hybrid glycan (N173 or N156), and the scaffolded V1-V2 loop; B12 recognizes the CD4 binding domain on gp120. Thus, a comprehensive physicochemical characterization of gp120's structure and post-translational modifications has enormous implications for HIV vaccine design and will provide insights for future integration of clinical data. The gp120 materials characterized in the current study were reference standard materials. They were used throughout the course of development and stability study activities and were highly representative of the clinical lots.

### Methods

### Production of Reference Lots

Gp120 reference materials were produced during consistency runs using animal component-free media in single use bioreactors. The cell culture process utilized commercially available or proprietary platform media for vial thaw, inoculum expansion and production process. Clarified cell culture harvest underwent multiple chromatography and filtration steps were used during purification to ensure the consistency of critical quality attributes of the antigen (antigenicity, purity, and yield) as well as the effective removing of impurities such as DNA, virus, and host cell proteins.

### Intact Molecular Weight Determination

Molecular weight (MW) of intact gp120s was measured by MALDI-TOF using a Bruker UltrafleXtreme MALDI-TOF/TOF instrument. MW of de-N-glycosylated gp120s was determined by LC-MS (liquid chromatography-mass spectrometry) using a Waters Xevo G2-S QTOF and the MaxEntl deconvolution software.

### Immunogenicity Assessment

CB6F1 mice (hybrid of C57B1/6 and Balb/C mice) were immunized intra-muscularly at days 0, 14, and 28 with 2 μg each of gp120 proteins without adjuvant or formulated with 50 μg aluminium hydroxide or 50 μl AS01. The AS01 used here, called AS01_{B}, was a liposome-based formulation containing 50 μg 3-O-desacyl-4′-monophosphoryl lipid A (MPL, GSK Vaccines, Rixensart, Belgium) and 50 μg QS-21 Stimulon® (Quillaja saponaria Molina, fraction 21. Licenced by GSK from Antigenics Inc, a wholly owned subsidiary of Agenus Inc, Lexington, Mass., USA) in 500 μl. The animals received 1/10^{th} of the human dose, which means that they received 5 μg MPL and 5 μg QS-21. Antibody responses were characterized at 14 days post-second and third dose and T cell responses were analysed at 7 days or 14 days post-second and third dose.

For leukocyte isolation, blood was collected and 5 pools of 7 mice/group were constituted before addition of RPMI/additives (RPMI 1640, supplemented with Glutamine, Penicillin/streptomycin, Sodium Pyruvate, non-essential amino-acids and 2-mercaptoethanol) containing heparin (1/10). Ten volumes of Lysing buffer were added to the whole blood and tubes were incubated at room temperature (RT) for 10 min. After centrifugation (400 g, 10 min at RT), the pellet was harvested in RPMI/additives and filtered (Cell strainer 100 μm). Cells were pelleted again (400 g, 10 min at RT) and resuspended in Complete Medium (RPMI 1640, supplemented with Glutamine, Penicillin/streptomycin, Sodium Pyruvate, non-essential amino-acids and 2-mercaptoethanol, and 5% Heat inactivated Fetal Calf Serum.

For splenocytes isolation, spleens were collected and placed in RPMI/additives (supplemented with Glutamine, Penicillin/streptomycin, Sodium Pyruvate, non-essential amino-acids and 2-mercaptoethanol). Cell suspensions were prepared from each spleen using a tissue grinder. The splenic cell suspensions were filtered (Cell strainer 100 μm). The filter was rinsed with 40 ml cold RPMI/additives. After centrifugation (1300 RPM, 10 min at RT), Cells were resuspended in Complete Medium (RPMI supplemented with Glutamine, Penicillin/streptomycin, Sodium Pyruvate, non-essential amino-acids and 2-mercaptoethanol, and 5% Heat inactivated Fetal Calf Serum).

Fresh pools of leukocytes or splenocytes were plated in round bottom 96-well plates at approximately 1 million cells per well. Cells were then stimulated for 6 hours (37° C., 5% CO₂) with anti-CD28 (clone 9C10 (MFR4.B) and anti-CD49d (clone 37.51) (BD Biosciences) at 1 μg/ml, with or without 5 μg/ml of 1086.C or TV1.C purified gp120 proteins. After a 2 hour-stimulation, Brefeldin A diluted 1/1000 in complete medium was added for 4 additional hours. Plates were then transferred at 4° C., overnight. Next, cells were stained and analyzed using a 5-colour ICS assay. Cells were transferred to V-bottom 96-well plates, centrifuged at 189 g for 5 min at 4° C. and resuspended in 50 μl Flow Buffer (PBS 1×, 1% FCS, 0.02% azide) containing anti-CD16/32 (clone 2.4G2) diluted 1/50 for 10 min at 4° C. Then, 50 μl Flow Buffer containing anti-CD4-V450 (clone RM4-5) and anti-CD8-PerCp-Cy5.5 (clone 53-6.7) antibodies (final dilution 1/50 each, BD Biosciences) and Live/dead-PO (1/500) was added for 30 min at 4° C. Cells were pelleted (189 g, 5 min, 4° C.), washed with 200 μL Flow Buffer, fixed and permeabilized by adding 200 μL of Cytofix/Cytoperm solution for 20 min at 4° C. (BD Biosciences, USA). Cells were centrifuged (189 g for 5 min at 4° C.) and washed with 200 μL Perm/Wash buffer (BD Biosciences, USA). After an additional centrifugation step, cells were stained in 50 μL Perm/Wash buffer with anti-IL2-FITC (clone JES6-5H4, 1/50), anti-IFNγ-APC (clone XMG1.2, 1/50) and anti-TNFα-PE (clone MP6-XT22, 1/700) antibodies (BD Biosciences), for 2 h at 4° C. Cells were washed twice with the Perm/Wash buffer harvested in 300 μL BD Stabilizing Fixative solution. Stained cells were analyzed by flow cytometry using a LSRII flow cytometer (BD Biosciences) and FlowJo software (Tree Star, Inc).

Anti-1086.C and anti-TV1.C gp120 binding antibodies were measured by ELISA. 96-well Elisa plates were coated with the 1086.C or TV1.C gp120 proteins (0.25 μg/ml or 0.5 μg/ml respectively). Sera from vaccinated mice were serially diluted and incubated for 1 hour at 37° C. Serial dilutions of the standard were used to calculate the anti-1086.C or TV1.C gp120 antibody standard titers of tested sera. Plates were washed with PBS 0.1% tween20 buffer after each incubation step. Peroxidase-AffiniPure Goat Anti-Mouse IgG (H+L) antibodies (1/4000) were added for 1 hour at 37° C. and after a washing step, the antigen-antibody complex was revealed by incubation with a peroxidase substrate ortho-phenylenediamine dihydrochlorid/H₂O₂ (15 min). The Optical densities (O.D.) were recorded at 490-620 nm. The anti-1086.C and anti-TV1.C gp120 antibody titers of individual animals were determined from the standard curve of the ELISA using a regression model. Geometric Mean Titers (GMT) with 95% confidence interval were then calculated for each group of mice.

Anti-gp70-V1V2 antibodies were measured using ELISA as described above, except that the 96-well Elisa plates were coated with the recombinant antigen gp70-V1V2 (gp70-V1V2 scaffold (Clade B/Case A2) (Haynes B F, McElrath G P M J, Zolla-Pazner S, Tomaras G D, Alam S M, Evans D T, et al. Immune-correlates analysis of an HIV-1 vaccine efficacy trial. N Engl J Med 2012; 366: 1275-1286)).

### Peptide Mapping and Isoelectric Focusing (IEF) Gel Electrophoresis

Gp120 proteins were denatured in Guanidine HCl, reduced by DTT, alkylated by lodoacetamide, de-N-glycosylated by PNGase F, and then analyzed by RP-HPLC using a C18 column. UV at 215 nm and MS/MS were used for online detection and identification. IEF was performed using the Invitrogen Novex Precast IEF gels (pH 3-7 and pH 3-10) and associated buffers.

### Differential Scanning Calorimetry (DSC) and Circular Dichriosm (CD)

DSC was performed using a Microcal VP-DSC scanning microcalorimeter. For CD, Gp120 materials were buffer exchanged into 10 mM phosphate at pH 7.0 and then analyzed by a Jasco J-1500 Circular Dichroism Spectrometer. The Contin/LL program in the CDPro Analysis software was used to deconvolute experimental spectra with reference to the SP43 dataset consisting of soluble proteins.

### O-Linked Glycosylation Site Mapping and Identification

Reduced, alkylated, and de-N-glycosylated tryptic peptides of gp120 proteins were analzyed by LC-MS/MS on Xevo G2-S operated under Product Ion Discovery (PID) mode. Briefly, the MS (mass spectrometer) was programmed to fragment and sequence all precursor ions that gave rise to signature sugar peaks (m/z 204.1 for HexNAc, m/z 366.1 for HexNAcHex, m/z 292.1 for NeuAc, m/z 274.1 for NeuAc-H₂O) upon collision. The identification of the O-linked glycans was based on accurate mass of the glycans.

### N-Linked Glycosylation Characterization

For N-linked glycosylation site mapping, reduced and alkylated tryptic peptides were digested by Endo H, Endo F3, or PNGase F, and then analyzed by LC-MS/MS using a Thermo LTQ Orbitrap mass spectrometer operated under data-dependent acquisition mode. Data were analyzed to look for variable modifications of GlcNAc at Asn residues. For N-linked glycoprofiling, gp120 proteins were heated at 90° C. in the presence of surfactant RapiGest SF (Waters) and then de-N-glycosylated by Rapid PNGase F (New England Biolab). Proteins were removed from the released N-glycans by ethanol precipitation. The purified glycans were labeled by 2-AB using reductive amination. Labeled glycans were resolved by LC using a Waters Acquity Glycan BEH Amide column with both fluorescent and MS detection. SimGlycan software (Premier Biosoft) was used to analyze the MS/MS data for glycan identification.

### Disulfide Bond Mapping

For disulfide bond analysis, gp 120 proteins were in-solution digested by trypsin with or without reduction/alkylation and then de-N-glycosylated by PNGase F. The peptides were extensively analyzed by the LC-MS/MS using LTQ Orbitrap with both Collision Induced Dissociation (CID) and Electron Transfer Dissociation (ETD). For detection of free, non-bonded Cys residues, gp120 was alkylated by lodoacetamide without prior reduction by DTT before trypsin and PNGase F digestion.

### Results and Discussion

### Comparison of the Reference Materials to CTM (Clinical Trial Material)

The development reference materials are different from the research materials, which was generated and used only in the early discovery phase. The reference materials described here were produced from the same parent cell line for cell bank, manufactured with similar upstream and downstream process, and stored in the same formulation buffer at the same temperature as the CTM. A panel of testing was performed, which showed they were similar in all aspects of critical quality attributes (CQA). These results are summarized in the following table.

TABLE A
Comparison of the reference materials to CTM

| | | 1086.C | | TV1.C | |
|---|---|---|---|---|---|
| Analytical Test | Units | CTM | Reference Material | CTM | Reference Material |
| Purity (RP-HPLC) | % | 98.5 | 95.2 | 99.3 | 96.7 |
| Monomer (SEC) | % | 95.7 | 96.4 | 87.9 | 83.1 |
| Intact Monomer | % | 85.7 | 89.0 | 91.9 | 92.5 |
| (Reduced SEC) | | | | | |
| CD4 binding (Biacore) | % | 87 | 100 | 98 | 91 |
| CHO Host Cell Protein | ng/ug | 2.4 | 15.8 | 7.3 | 21.4 |
| (ELISA) | gp120 | | | | |
| Residual DNA (qPCR) | ng/100 | <0.001 | <0.001 | <0.001 | <0.001 |
| | ug | | | | |
| | gp120 | | | | |
| pH | | 6.9 | 7.0 | 6.3 | 6.3 |
| Appearance | | clear, colorless, | clear, colorless, | clear, colorless, | clear, colorless, |
| | | free of visible | free of visible | free of visible | free of visible |
| | | particulates | particulates | particulates | particulates |

### Intact MW, Charge Heterogeneity, Higher Structure, and Melting Point

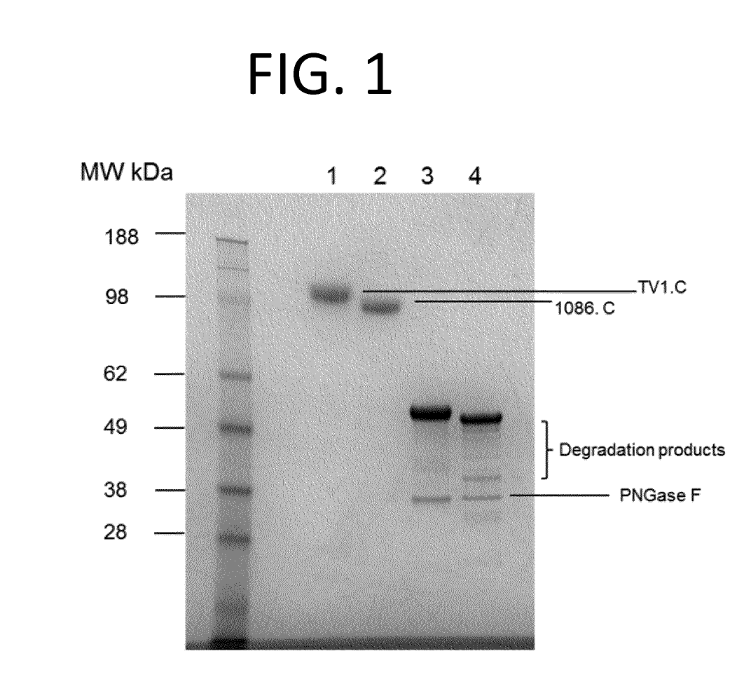

The gp120 name comes from the apparent MW of approximately 120 KDa from band mobility on the SDS-PAGE gels. Gp120s are heavily glycosylated with N-linked glycans contributing approximately half the molecular mass. A reduced SDS-PAGE gel analysis of both neat and de-N-glycosylated TV1.C and 1086.C gp120 is shown in FIG. 1. Indeed, the apparent MW of TV1.C gp120 was reduced ˜50% after de-N-glycosylation by Peptide-N-Glycosidase F (PNGase F). The presence of lower MW bands was due to clipping by proteases (discussed later) during prolonged incubation at 37° C. Gel mobility can be affected by many factors, such as post-translational modifications and matrix effects. Thus, the apparent MW may not be a true indication of the molecular mass. To better determine MW, mass spectrometric methods were used. Intact neat gp120s were hard to resolve by LC-MS likely due to complexity of glycosylation. Thus, it was analyzed by MALDI-TOF instead. The average MW of TV1.C and 1086.C gp120s was determined to be 105,041.8 Da and 94,938.7 Da, respectively. The de-N-glycosylated gp120s were analyzed by LC-MS. After deconvolution, the MW of the main species was 57,965 Da for TV1.C and 52,823 Da for 1086.C (data not shown). Thus, in both molecules, glycans accounted for ˜45% of the molecular mass. Besides, multiple smaller peaks were also observed with Δmass of 294 Da and 656 Da, which corresponded to the mass of mono- and oligo-saccharides, and suggested the presence of O-linked glycans on gp120 molecules.

The calculated isoelectric points (pI) of gp120s were slightly basic above 8. But due to extensive glycosylation, many of which are acidic glycans, the pI was expected to be acidic. This was confirmed by IEF gel analysis. Also, due to the overwhelming complexity of glycosylation, gp120s exhibited charge heterogeneity that exceeded the resolving capability of a regular IEF gel. Overall, gp120s contained species with pI within 3.5-5.2. TV.C gp120 seemed to have a broader pI range than 1086.C.

To gain a low-resolution characterization of gp120 secondary and tertiary structures and set a benchmark for comparison among lots, Circular Dichroism (CD) analysis was performed using near- and far-UV regions. The two gp120 molecules clearly showed different CD spectra in both near- and far-UV regions, suggesting that the chronic form had evolved into slightly different tertiary and secondary structures than the early transmitted form. The main differences were more α-helix and less β-strand in 1086.C than in TV1.C gp120. Interestingly, it has been reported that aligned sequences of 106 HIV isolates and found intrinsic variations in propensities toward different secondary structures in the V1V2 regions and the propensities correlated with binding to different bNAbs.

We used Differential Scanning Calorimetry (DSC) to characterize the thermodynamics of gp120s. The protein melting point (Tm) in a given solvent environment, which is indicative of protein unfolding, is a commonly used measurement of thermal stability of proteins. TV1.C gp120 showed thermal transitions that spanned a wide temperature range with Tm at 61.2° C. In contrast, 1086.C gp12 showed a sharp and strong main peak transition and a higher Tm at 63.7° C. The difference suggested 1086.C gp120 had a tighter-packed and better-defined structure than TV1.C.

Immunogenicity of gp120s

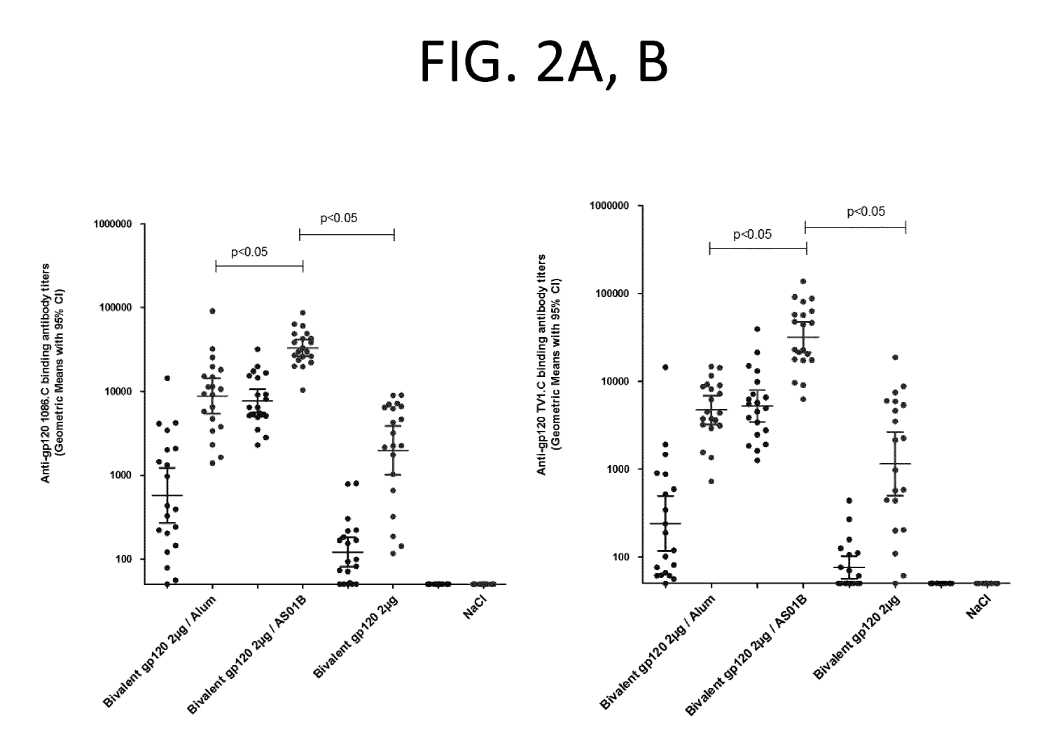

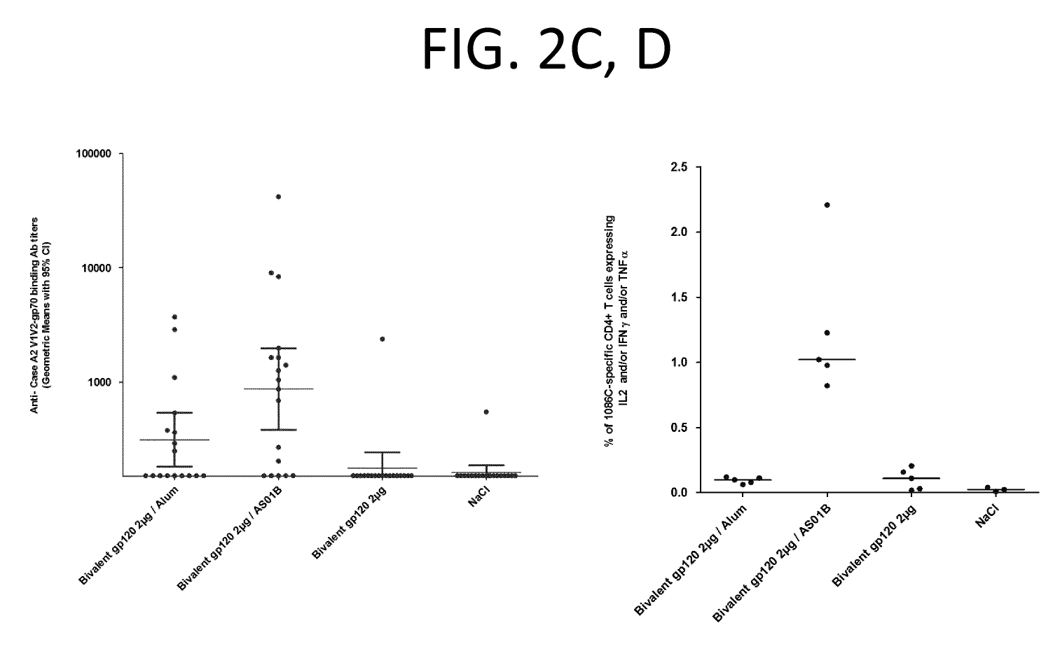

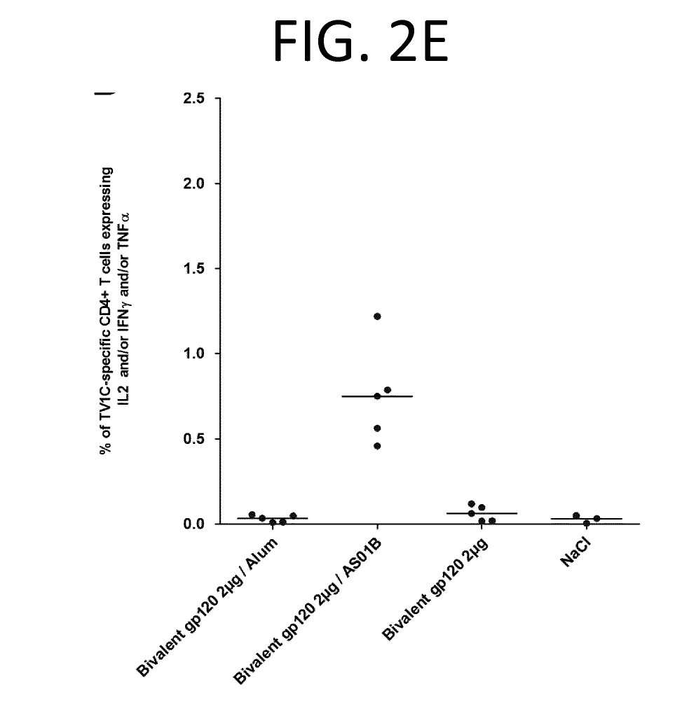

Non-adjuvanted bivalent 1086.C & TV1.C gp120 antigens elicited detectable but low levels of binding antibodies with geometric mean titers (GMT) of 1973 and 1145, respectively, at 14 days post-third dose. Aluminium hydroxide adjuvanted gp120 antigens significantly increased binding antibody titers up to 8807 (anti-1086.C GMT) and 4698 (anti-TV1.C GMT). AS01-based formulation elicited the highest binding antibody responses reaching anti-1086.C and anti-TV1.C binding antibody titers of 32936 and 31860, respectively (FIGS. 2A and 2B). Post-third immunization, cross-reactive anti-V1V2 binding antibody responses (gp70-V1V2 scaffold Clade B/Case A2) were detected with the strongest titers measured when the bivalent 1086.C & TV1.C gp120 antigens were formulated with AS01, although some animals remained negative (FIG. 2C). Very low to non-detectable 1086.C- and TV1.C-specific CD4+ T cell responses were measured at 14 days post-third immunization with the bivalent Clade C gp120 antigens alone or adjuvanted with Aluminium hydroxide. In contrast, the gp120s/AS01 formulation elicited robust 1086.C- and TV1.C-specific CD4+ T cell responses (medians of 1% and 0.75% respectively) 14 days post-third dose (FIGS. 2D and 2E). Together, these data showed that the bivalent Clade C gp120 antigens formulated with the AS01 Adjuvant System elicited potent 1086.C & TV1.C gp120-specific antibody and CD4+ T cell responses in CB6F1 mice.

### Primary Sequence and Peptide Mapping

The primary amino acid sequences deduced from corresponding cDNA sequences are given by SEQ ID NO:1 and 2. TV1 gp120 contains 488 residues; 1086.C gp120 contains 469 residues. Since gp120s are heavily glycosylated and the added heterogeneity of glycans complicates the peptide maps, gp120 tryptic peptides were de-N-glycosylated before peptide mapping experiment was run. UV 215 was used as the detection method for the chromatography; MS/MS was used for identification of the peptide peaks. Through an 80 min LC, a sequence coverage of 92.6% (based on amino acid numbers) was achieved for TV1.C and 96.6% for 1086.C. With peptide mapping, a number of peptides originating from endogenous clipping were observed. In 1086.C gp120, the most abundant clipping occurred within ²⁸⁶IRIGPGQTFYATG²⁸⁰ (SEQ ID NO:3), which was in V3 loop of gp120. Similar cleavage in TV1 gp120 was also observed but at much reduced level. Besides V3 loop, less significant clipping near C5 domain was also observed in both gp120 molecules. Cleavage of gp120 by serine proteases is well known and extensively documented in the literature. Interestingly, trace amounts of several host cell proteases (Cathepsin Z, B, D, and A) co-purified with 1086.C gp120, while Cathepsin A co-purified with TV1 gp120. Cathepsin-induced degradation has also been reported for other recombinant protein expressed in CHO cells. Since Cathepsins have optimum activities under acidic condition, measures were taken to minimize and control gp120 clipping during manufacturing process and formulation. No clipping was detected in V1-V2 domain of gp120s, which is important for bNAbs PG9/PG16 recognition. Also of note, it was found that two Met residues (Met67 and 71 in 1086.C; Met71 and 75 in TV1.C) were prone to oxidation under oxidative condition. These Met residues are within the CD4 binding domain. Oxidation at these sites coincided with impaired CD4 binding by Biacore assay (data not shown), which presumably would lower the immunogenicity of the immunogens. This suggested the importance of minimizing oxidative stress and monitoring oxidation level at the CD4 binding domain.

### O-Linked Glycosylation Characterization

LC-MS of de-N-glycosylated TV1.C and 1086.C gp120s also suggested presence of O-glycans (discussed above). To map the exact site(s) of O-linked glycosylation and to characterize the O-glycan(s), a Product Ion Discovery (PID) based mass spectrometric approach was used with a Q-TOF MS. The MS was set to search for de-N-glycosylated peptides that generated the signature sugar peaks upon Collision Induced Dissociation (CID) and target those peptides for sequencing. Three peptides ¹NTEDLWVTVYYGVPVWR¹⁸ (SEQ ID NO:4), ⁴⁰²MWQGVGQATYAPPIAGNITCR⁴²² (SEQ ID NO:5), ⁴⁶⁵VVEIKPLGIAPTKAK⁴⁷⁹ (SEQ ID NO:6) in TV1.C gp120 and two peptides ¹SWVTVYYGVPVWK¹³ (SEQ ID NO:7), ⁴⁴⁴YKVVEIKPLGVAPTEAKR⁴⁶¹ (SEQ ID NO:8) in 1086.C gp120 were found to bear O-linked glycans. Since 1086.C peptide ⁴⁴⁴YKVVEIKPLGVAPTEAKR⁴⁶¹ (SEQ ID NO:8) and TV1.C peptide ⁴⁶⁵VVEIKPLGIAPTKAK⁴⁷⁹ (SEQ ID NO:6) each contains only one Serine or Threonine residue, the O-linked glycan could only be on T457 and T476, respectively. Either S1 or T4 in 1086.C peptide ¹SWVTVYYGVPVWK¹³ (SEQ ID NO:7) could be the potential site of O-linked glycosylation. CID from the Q-TOF MS was not able to differentiate the two sites since O-linked glycosidic bonds were labile under CID condition and completely fell off before the peptide backbone was fragmented. Alternatively, Electron Transfer Dissociation, a mild fragmentation technique that preserves the labile glycosidic bonds, was used to specifically target the precursor ion and pinpointed T4 as the O-linked glycosylation site. Either T2 or T8 in peptide ¹NTEDLWVTVYYGVPVWR¹⁸ (SEQ ID NO:4) could be the potential site of O-linked glycosylation. MS was not able to pinpoint the exact site of modification. Based on sequence homology with T4 in ¹SWVTVYYGVPVWK¹³ (SEQ ID NO:7) of 1086.C gp120, T8 was predicted as O-glycosylation site in TV1.C gp120. For peptide ⁴⁰²MWQGVGQATYAPPIAGNITCR⁴²² (SEQ ID NO:5), since N418 was identified as being modified by N-glycan (discussed in later section), T420 was unlikely to be modified by O-glycan due to steric hindrance. Thus, T410 was the predicted site of O-linked glycosylation. All the detected O-glycans were predicted to have a Core 1 mono- or di-sialylated GalNAc-Gal structure based on accurate mass. O-glycosylation near C-terminal sequence of gp120 was previously reported. The current study was the first to report O-glycosylation near N-terminal end of gp120 sequence. More interestingly, gp120 from the chronic form of HIV virus obtained a new O-glycosylation site T410 in C4 domain. The corresponding site on 1086.C gp120 is not occupied by a Thr residue. The function and immunological implication of O-glycans on gp120s remain largely unknown and await future investigation.

### N-Linked Glycosylation Characterization

Gp120s are heavily N-glycosylated with ˜45% mass accounted for by glycans. TV1.C and 1086.C gp120s have 30 and 23, respectively, of potential N-linked glycosylation sites (PNGS), which fit the N-linked glycosylation consensus motif (N-X-S/T, X being any amino acid but Pro). To map the exact sites of modification, an approach that combined LC-MS/MS analysis and endoglycosidase treatment was used. Two endoglysosidases Endo F3 and Endo H, which respectively cleave between the two core GlcNAc on complex N-glycans and high mannose/hybrid glycans leaving only one GlcNAc still attached to the Asn residue, was used. The reasons to use such treatment were two-fold: one is to reduce the complexity of the N-glycans and make MS/MS data easier to interpret; the other is to differentiate sites with complex or high mannose/hybrid glycans. By comparing the endoglysosidase treated samples with the non-treated, and PNGase F treated samples, we were able to obtain the overall N-glycosylation schemes in gp120s (FIG. 3A). In TV.1C gp120, 29 of the 30 PNGS were modified with 7 being exclusively modified by complex glycans, 7 being exclusively modified by high mannose/hybrid glycans, and 4 being modified by both complex and high mannose/hybrid glycans. Ten sites being fully occupied by glycans, and 19 sites were partially modified. Of note, N334 was not modified at all although it is a PNGS. Some N418 was found to be modified by a single HexNAc residue, which was not common but also has been reported previously. In 1086.C gp120, all 23 PNGS were modified with 5 being exclusively modified by complex glycans, 9 being exclusively modified by high mannose/hybrid glycans, and 4 being modified by both complex and high mannose/hybrid glycans. Nine sites were fully occupied by glycans, and 14 sites were partially modified. Similarly, some N157, N367, N404 were found to be modified by a single HexNAc residue. From these results, it was clear that gp120 from chronic form TV1.C had evolved to obtain more N-glycosylation sites and increased complexity. Since relative percentages of glycans remained the same (˜45%, data shown in earlier section) and the numbers of fully glycosylated sites were more in TV1C gp 120 than in 1086.C gp120, it was likely that the chronic form evolved to bear more high mannose/hybrid glycans, which are overall smaller in MW. The dataset also confirmed the presence of high mannose glycan clusters around C2-V3-C3-V4-C4 domains in both molecules, which were known as epitopes for bNAb 2G12 and partial epitope for PGT128.

Glycosylation profiles of the gp120s were characterized by combining fluorescence labeling of the released N-glycans and HPLC separation with both fluorescence and MS/MS detection. As expected, high mannose and complex type (sialylated bi-, tri-, and tetra-antennary) glycans were the main species detected in the gp120 molecules (FIG. 3B). The dense glycan population on the surface of HIV-1 envelope spike, primarily attributed to the gp120 proteins, was considered the ‘silence face’ that shielded the virus from immune recognition. Indeed, gp120 glycans are processed solely by host cell glycosylation machinery. Cross-reactivity to glycans present on HIV-1 envelope spike and on host cell proteins leads to the intrinsic low immunogenicity of HIV-1 viral glycans. One unique feature of HIV-1 Env glycosylation is the clusters of oligomannose glycans, which is highly conserved across all HIV-1 clades but not usually seen in primate host cell proteins. In fact, a large fraction of the known bNAbs recognize HIV-1 virus by selectively targeting high mannose glycans on gp120, for examples PGT125-130, PGT141-145, and CH01-CH05. Thus, the oligomannose clusters have tremendous implications in vaccine designs. It was previously reported that recombinant monomeric gp120 expressed from 293T cells bore only ˜30% oligomannose, significantly lower than virion-associated gp120s from primary virus (62-79%). From the glycoprofiling experiments, percentages of oligomannose in TV1. C and 1086.C gp120s were determined at 55.5% and 57.2%, respectively. This indicated that our recombinant monomeric gp120s had comparable oligomannose contents as virion-associated gp120s.

### Disulfide Bond Characterization

TV1.C and 1086.C gp120s each contain 18 cysteine residues, which form intra-molecular disulfide bonds and stabilize the tertiary structure. Correct disulfide bonding is critical in maintaining the structural integrity. Heterogeneity has been reported in the literature for several gp140 proteins that were recombinantly produced. It was noticed that both TV1.C and 1086.C gp120 materials contained a dimer band upon non-reduced SDS-PAGE gel analysis, while the band completely disappeared upon reduced SDS-PAGE. It was suspected that dimers were formed through inter-molecular disulfide bonding. To map the disulfide bonds, extensive LC-MS/MS analysis using both electron transfer dissociation (ETD) and collision induced dissociation (CID) was performed on the deglycosylated gp120 tryptic peptides before and after reduction with DTT. An intermolecular disulfide bond was detected between two identical peptides ⁴⁰²MWQGVGQATYAPPIAGNITCR⁴²² in TV1.C gp120 and ¹⁷TTLFCASDAK²⁶ (SEQ ID NO:9) in 1086.C gp120, which contributed to the formation of dimer species. In addition, analysis of the alkylated protein without prior DTT reduction showed readily detectable amount of free Cys residues in at least two peptides (¹⁷TTLFCASDAK²⁶ (SEQ ID NO:9) and ³⁸⁹AIYAPPIEGEITCNSNITGLLLLR⁴¹²) (SEQ ID NO:10) in 1086.C gp120. Clearly, the un-bonded Cys residues were also subject to inter-molecular disulfide bonding. Overall, disulfide bonding patterns delineated from the extensive LC-MS/MS studies were shown in both expected and alternative disulfide bondings were detected and are shown in FIG. 4. The data were in agreement with previous report that the disulfide heterogeneity was mostly in V1-V2 loop and flanking regions.

### Example 2: Immunogenicity Studies

### Dose-Range Immunogenicity Study of Bivalent Clade C Gp120 (TV1.C and 1086.C)/AS01B in Mice

The objective of this experiment was to assess the dose response relationship of bivalent Clade C gp120 (1086.C & TV1.C) antigens in CB6F1 mice (hybrid of C57BI/6 and Balb/C mice) when formulated in combination with AS01_{B} Adjuvant Systems, in terms of antigen-specific cellular and antibody responses. Animals were immunized intramuscularly on day 0, 14 and 28 with 10 μg, 2 μg, 0.4 μg or 0.08 μg of bivalent clade C gp120 antigens formulated with 50 μl of AS01_{B}. The induced T cell and antibody responses were characterized at 7 and 14 days post-third dose respectively.

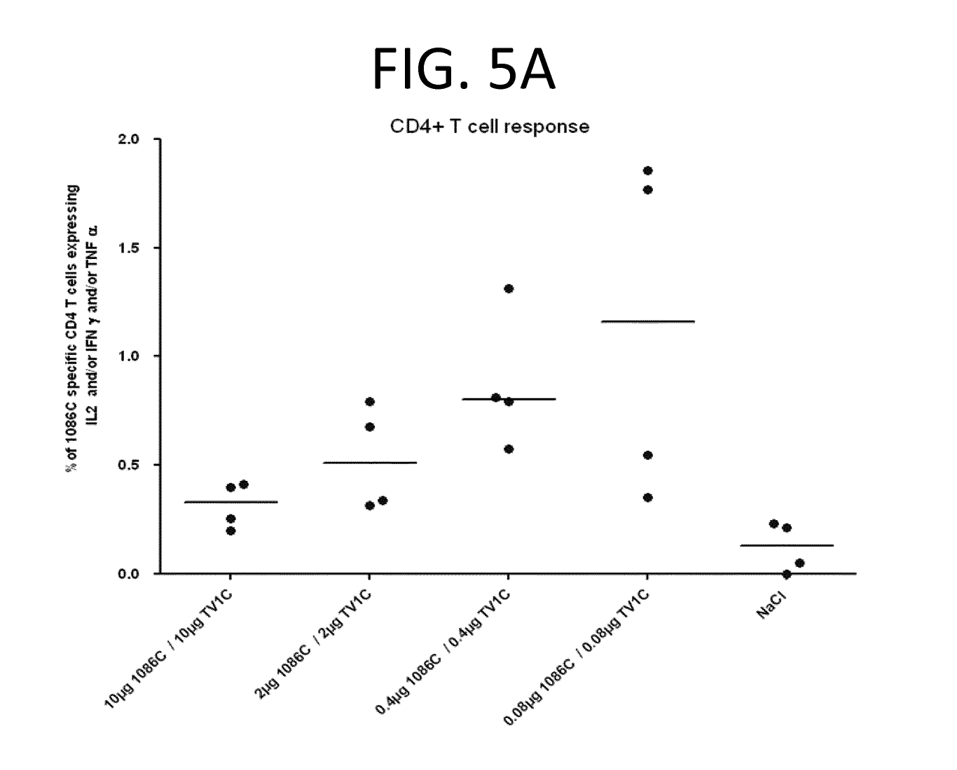

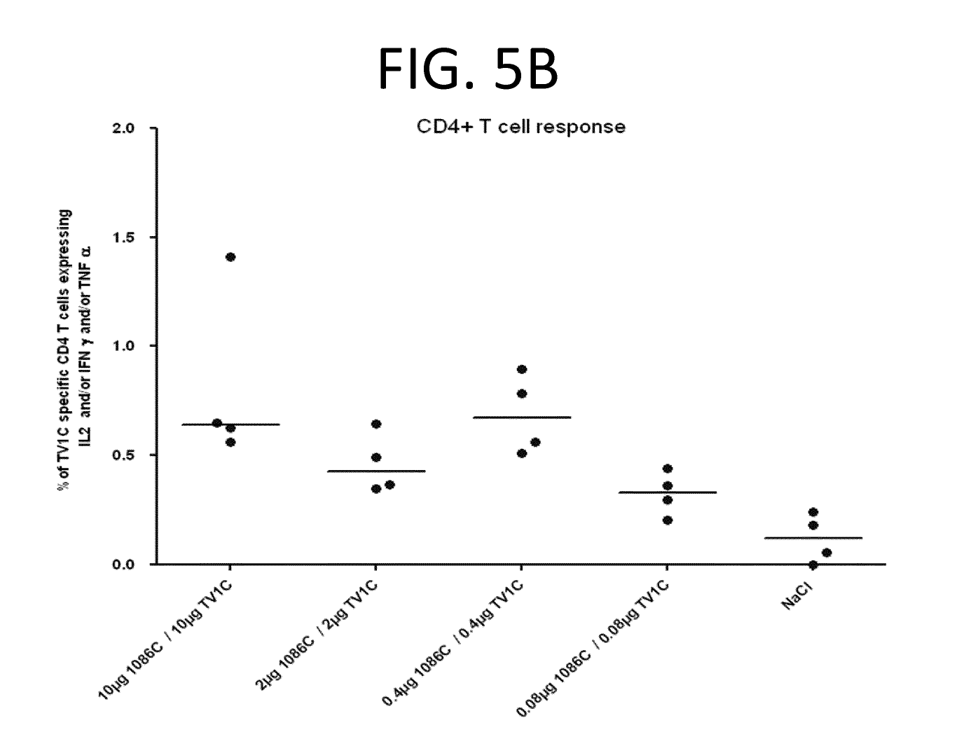

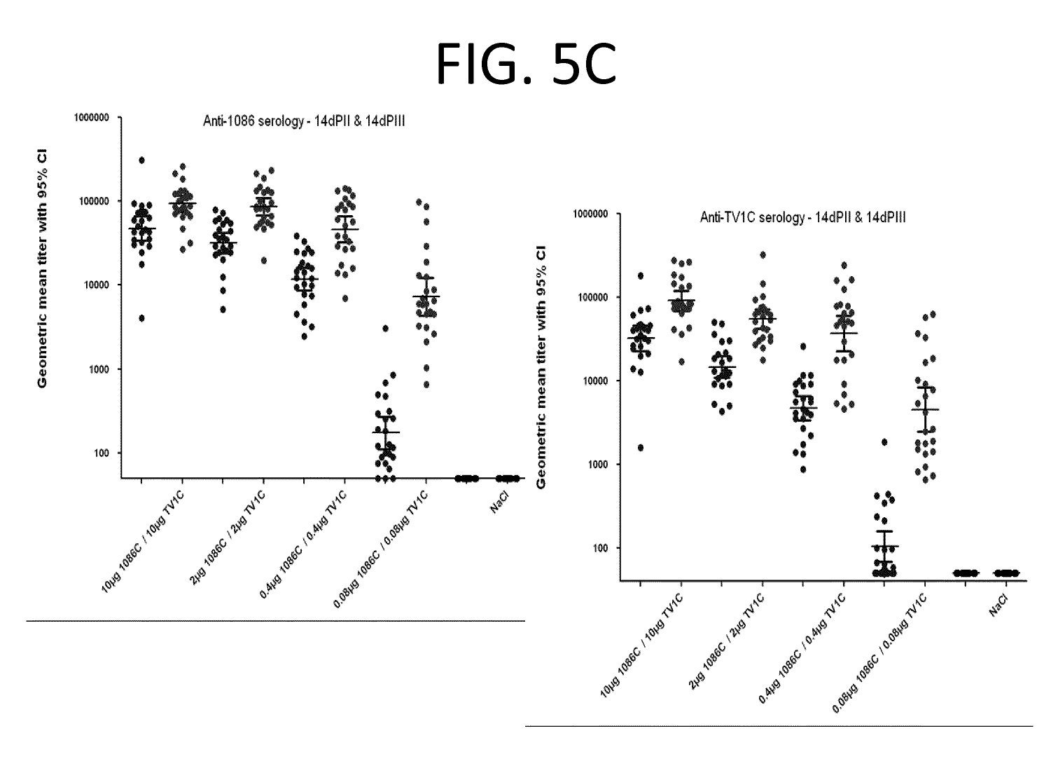

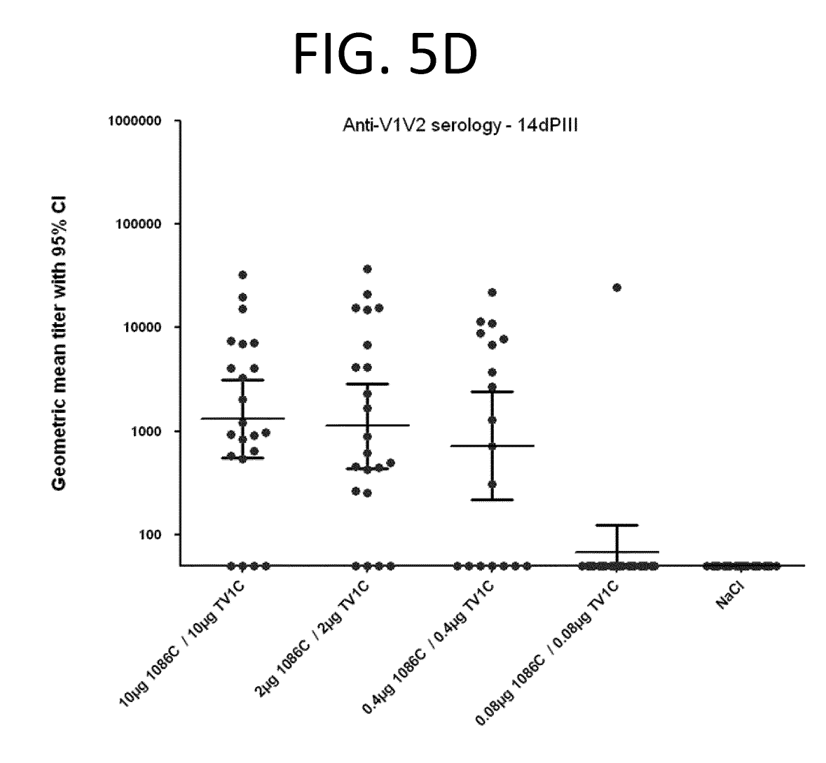

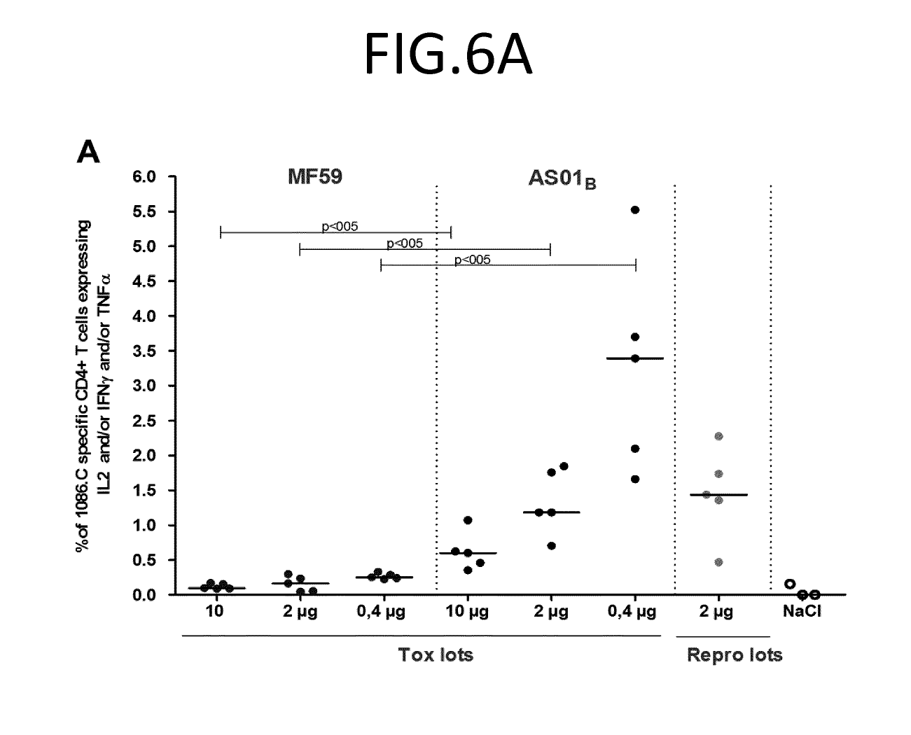

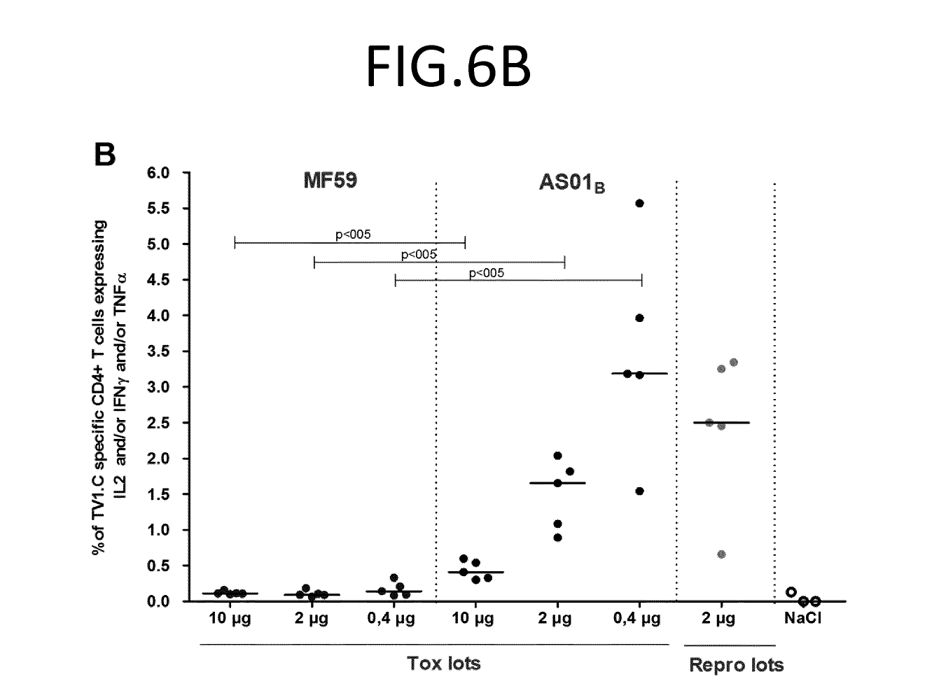

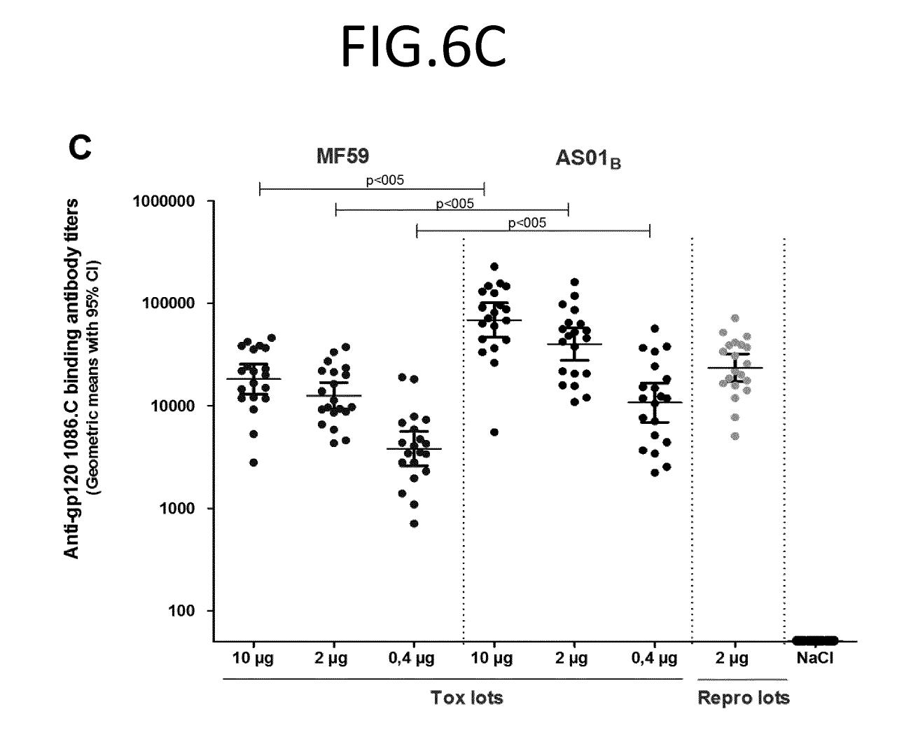

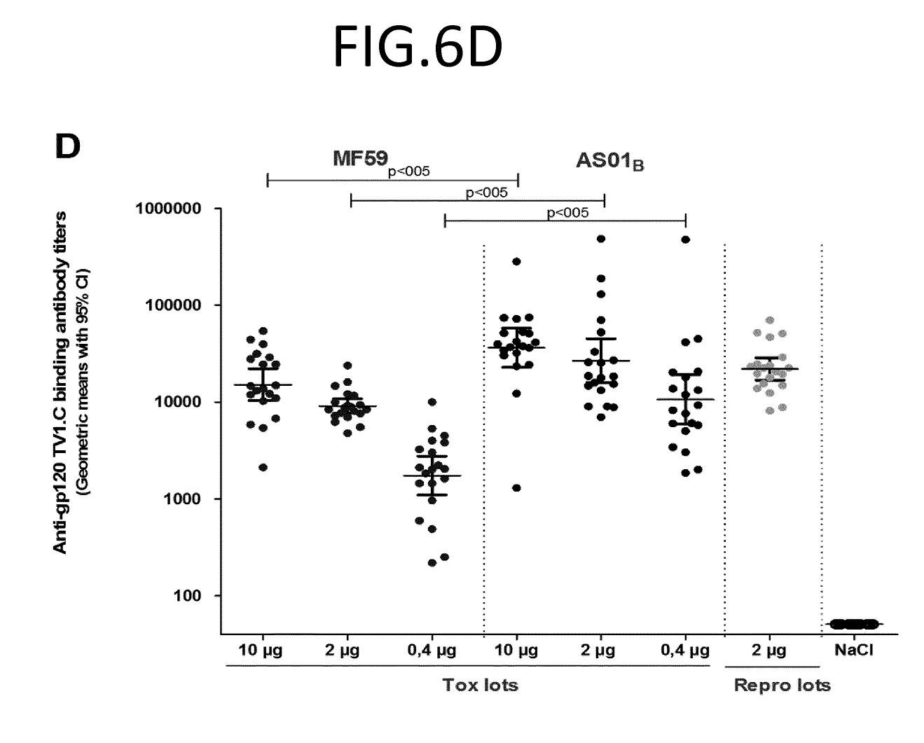

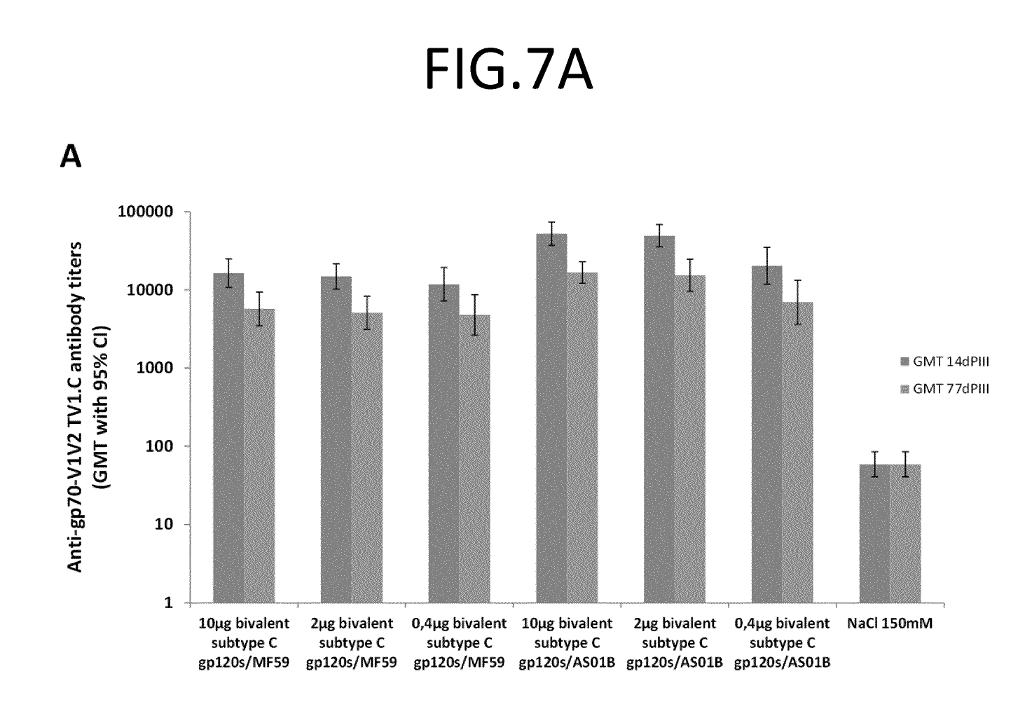

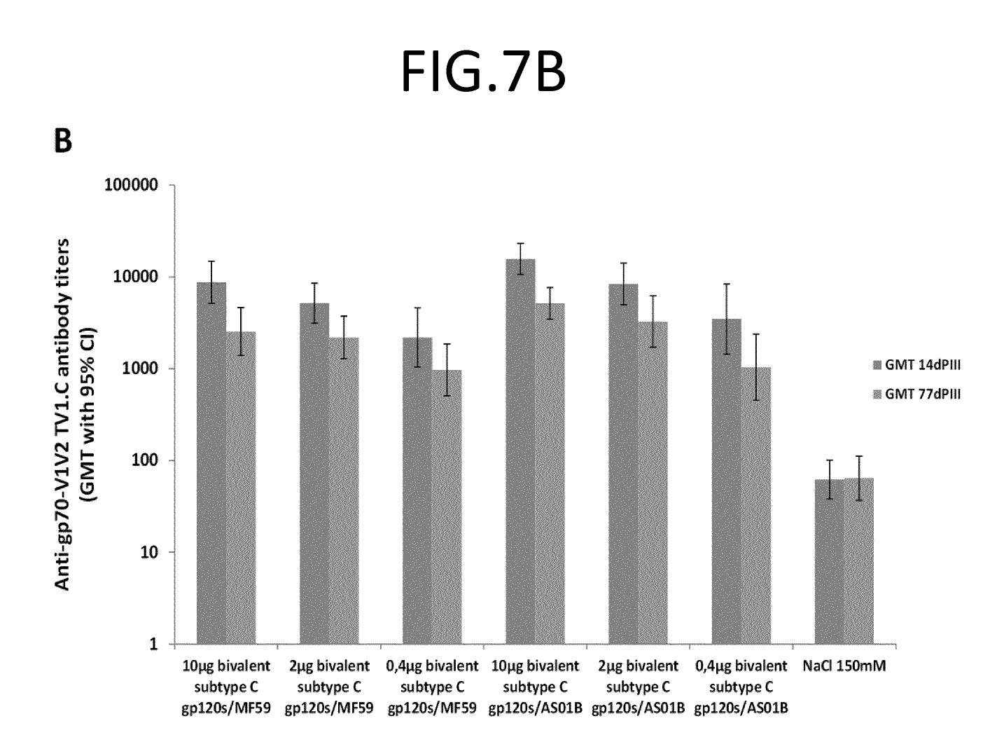

The bivalent clade C gp120/AS01_{B} vaccine formulation elicited 1086.C and TV1.C-specific CD4+ T cell response for all tested bivalent doses. No statistical differences were observed between doses, however, a trend for higher 1086.C-specific CD4+ T cell responses was observed with lower dose of bivalent Clade C gp120/AS01_{B} (FIG. 5A). This was not observed when measuring the TV1C-specific CD4+ T cell responses (FIG. 5B). The vaccine-induced CD8+ T cell responses at 7 days after 3^{rd} immunization were low to undetectable for both 1086.C & TV1.C responses (data not shown). The bivalent TV1.C and 1086.C gp120 antigens adjuvanted in AS01_{B} induced dose-dependent high level of anti-1086.C and TV1C antibody responses at 14 days post 2^{nd} and 3^{rd} immunization (FIG. 5C). Moreover, for all antigen doses tested, similar levels of 1086.C and TV1.C specific total Ig responses were observed, suggesting that there is no negative impact on the humoral responses when combining 1086.C and TV1.C gp120 antigens in AS01_{B} adjuvant system. Anti-V1V2 total Ig responses were also detected by an ELISA coated with the gp70-V1V2 scaffold (Clade B/Case A2) (Haynes et al., JEM 2012) (FIG. 5D).

Head to Head Preclinical Comparison of MF59 vs AS01_{B} to Formulate the Bivalent Clade C gp120s

The immunogenicity of the bivalent 1086.C & TV1.C gp120 Tox lots was characterized following immunization of CB6F1 mice (hybrid of C57BI/6 and Balb/C mice) with a dose range of gp120 antigens (0.4 μg, 2 μg or 10 μg each), formulated with either 50 μl MF59 (squalene-based oil-in-water emulsion) or 50 μl AS01B. As a benchmark, mice were immunized with 2 μg of the consolidation lots of the bivalent Clade C gp120s formulated with 50 μl AS01B. Animals received intramuscular injections at days 0, 14 and 28, and the T cell and antibody responses were monitored at 7 days or 14 days post-third dose respectively. In addition, anti-V1V2 antibody responses were monitored at 77 days post-third dose.

The bivalent clade C gp120s formulated in AS01B elicited potent 1086.C- and TV1.C-specific CD4+ T cell responses according to an inverse dose-range, suggesting that a high amount of antigen may trigger regulatory mechanisms leading to a decreased intensity of the induced-CD4+ T cell responses (FIGS. 8A and 8B). In contrast, very low to undetectable gp120-specific CD4+ T cell responses were measured following immunizations with the bivalent Clade C gp120s formulated in MF59.

The bivalent V1.C and 1086.C gp120 antigens formulated in AS01_{B} or MF59 induced dose-dependent high levels of anti-1086.C and “V1C antibody responses at 14 days post 3” immunization (FIGS. 8C and 8D). The intensities of both anti-1086.C and TV1C antibody responses were statistically significantly higher after immunization with the AS01_{B}-based formulations as compared to MF59-based formulations at all gp120 doses tested.

All together these data show that the bivalent Clade C gp120 antigens (1086.C & TV1.C) formulated with the AS01_{B} Adjuvant System elicit potent 1086.C & TV1.C gp120-specific antibody and CD4+ T cell responses with higher intensities than MF59-based formulations in CB6F1 mice.

Anti-gp70-V1V2 Subtype C antibodies were measured using ELISA. 96-well Elisa plates were coated with the recombinant antigen gp70-V1V2 TV1.C or gp70-V1V2 1086.C at 0.25 μg/ml and after a blocking step 1 hour at 37° C. with PBS BSA 1%/Tween 20 0.1%/NBC serum 4%, sera from vaccinated mice were serially diluted and incubated for 1 hour at 37° C. Plates were washed 4 times with PBS 0.1% tween20 buffer. A biotinylated anti-mouse antibody Ig Tot was then added (at 1/2000 for the TV1.C coating and 1/4000 for the 1086.C coating, Dako) for 1 hour at 37° C. and after a washing step and the antigen-antibody complex was revealed by incubation with a streptavidin horseradish peroxidase complex (30 min at 37° C.) and a peroxidase substrate ortho-phenylenediamine dihydrochlorid/H₂O₂ (20 min at Room temperature). The reaction was stopped with H₂SO₄ 1 M. The Optical densities (O.D.) were recorded at 490-620 nm. The anti-V1V2 antibody titer of each individual mouse serum was determined from the standard curve of the ELISA using a regression model. Geometric Mean Titers (GMT) with 95% confidence interval were then calculated for each group of mice.

These data show that the bivalent Clade C gp120 antigens (1086.C & TV1.C) formulated with the AS01_{B} Adjuvant System elicit anti-V1V2 antibody responses with higher intensities than MF59-based formulations in CB6F1 mice.

It should be understood that for all numerical bounds describing some parameter in this application, such as “about,” “at least,” “less than,” and “more than,” the description also necessarily encompasses any range bounded by the recited values. Accordingly, for example, the description “at least 1, 2, 3, 4, or 5” also describes, inter alia, the ranges 1-2, 1-3, 1-4, 1-5, 2-3, 2-4, 2-5, 3-4, 3-5, and 4-5, et cetera.

For all patents, applications, or other reference cited herein, such as non-patent literature and reference sequence information, it should be understood that they are incorporated by reference in their entirety for all purposes as well as for the proposition that is recited. Where any conflict exists between a document incorporated by reference and the present application, this application will control.

Headings used in this application are for convenience only and do not affect the interpretation of this application.

Preferred features of each of the aspects provided by the invention are applicable to all of the other aspects of the invention mutatis mutandis and, without limitation, are exemplified by the dependent claims and also encompass combinations and permutations of individual features (e.g., elements, including numerical ranges and exemplary embodiments) of particular embodiments and aspects of the invention, including the working examples. For example, particular experimental parameters exemplified in the working examples can be adapted for use in the claimed invention piecemeal without departing from the invention. For example, for materials that are disclosed, while specific reference of each of the various individual and collective combinations and permutations of these compounds may not be explicitly disclosed, each is specifically contemplated and described herein. Thus, if a class of elements A, B, and C are disclosed as well as a class of elements D, E, and F and an example of a combination of elements A-D is disclosed, then, even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-groups of A-E, B-F, and C-E are specifically contemplated and should be considered disclosed from disclosure of A, B, and C; D, E, and F; and the example combination A-D. This concept applies to all aspects of this application, including elements of a composition of matter and steps of method of making or using the compositions.

The forgoing aspects of the invention, as recognized by the person having ordinary skill in the art following the teachings of the specification, can be claimed in any combination or permutation to the extent that they are novel and non-obvious over the prior art—thus, to the extent an element is described in one or more references known to the person having ordinary skill in the art, they may be excluded from the claimed invention by, inter alia, a negative proviso or disclaimer of the feature or combination of features.

Sequence listing

SID:1, Amino acid sequence for TV1 gp120:
NTEDLWVTVYYGVPVWRDAKTTLFCASDAKAYETEVHNVWATHACVPTDP
NPQEIVLGNVTENFNMWKNDMADQMHEDVISLWDQSLKPCVKLTPLCVTL
NCTDTNVTGNRTVTGNSTNNTNGTGIYNIEEMKNCSFNATTELRDKKHKE
YALFYRLDIVPLNENSDNFTYRLINCNTSTITQACPKVSFDPIPIHYCAP
AGYAILKCNNKTFNGTGPCYNVSTVQCTHGIKPVVSTQLLLNGSLAEEGI
IIRSENLTENTKTIIVHLNESVEINCTRPNNNTRKSVRIGPGQAFYATND
VIGNIRQAHCNISTDRWNKTLQQVMKKLGEHFPNKTIQFKPHAGGDLEIT
MHSFNCRGEFFYCNTSNLFNSTYHSNNGTYKYNGNSSSPITLQCKIKQIV
RMWQGVGQATYAPPIAGNITCRSNITGILLTRDGGFNTTNNTETFRPGGG
DMRDNWRSELYKYKVVEIKPLGIAPTKAKRRVVQREKR
SID2: Amino acid sequence for 1086.C gp120:
SWVTVYYGVPVWKEAKTTLFCASDAKAYEKEVHNVWATHACVPTDPNPQE
MVLANVTENFNMWKNDMVEQMHEDIISLWDESLKPCVKLTPLCVTLNCTN
VKGNESDTSEVMKNCSFKATTELKDKKHKVHALFYKLDVVPLNGNSSSSG
EYRLINCNTSAITQACPKVSFDPIPLHYCAPAGFAILKCNNKTFNGTGPC
RNVSTVQCTHGIKPVVSTQLLLNGSLAEEEIIIRSENLTNNAKTIIVHLN
ESVNIVCTRPNNNTRKSIRIGPGQTFYATGDIIGNIRQAHONINESKWNN
TLQKVGEELAKHFPSKTIKFEPSSGGDLEITTHSFNCRGEFFYCNTSDLF
NGTYRNGTYNHTGRSSNGTITLQCKIKQIINMWQEVGRAIYAPPIEGEIT
CNSNITGLLLLRDGGQSNETNDTETFRPGGGDMRDNWRSELYKYKVVEIK
PLGVAPTEAKRRVVEREKR

1-20. (canceled)

21. A composition comprising two or more different human immunodeficiency virus (HIV) clade C envelope gp120 polypeptide antigens and a liposome-based adjuvant.

22. The composition of claim 21, wherein the two polypeptide antigens are less than 95% identical to each other, such as less than: 95, 94, 93, 92, 91, 90, 89, 88, 87, 86, 85, 84, 83, 82, 81, or 80% identical to each other.

23. The composition of claim 21, wherein the HIV clade C envelope gp120 polypeptide antigens exhibit a glycosylation pattern substantially as shown in FIG. 3A, a disulfide pattern substantially as shown in FIG. 4, or a glycosylation pattern substantially as shown in FIG. 3A and a disulfide pattern substantially as shown in FIG. 4.

24. The composition of claim 21, wherein the HIV clade C envelope gp120 polypeptide antigens comprise, consist essentially of, or consist of an amino acid sequence having at least: 70, 75, 80, 85, 90, 91, 92, 93, 94, 95, 96, 97, 98, 99%, or more, identity to a sequence selected from SEQ ID NO: 1, SEQ ID NO:2, or SEQ ID NO:1 and 2, such as at least 95, 96, 97, 98, 99, 99.1, 99.2, 99.3, 99.4, 99.5, 99.6, 99.7, 99.8, 99.9% identity to SEQ ID NO: 1, SEQ ID NO:2, or SEQ ID NO: 1 and 2.

25. The composition of claim 21, wherein the liposome-based adjuvant comprises a phosphatidylcholine (PC) and a sterol.

26. The composition of claim 25, wherein the PC is 1,2-dioleoyl-sn-glycero-3-phosphocholine (DOPC or the sterol is cholesterol, or the PC is DOPC and the sterol is cholesterol.

27. The composition of claim 25, further comprising a lipophilic or amphipathic immunostimulant, optionally wherein the immunostimulant is selected from a monophosphoryl lipid A (MPL), a saponin or an MPL and a saponin, optionally wherein the saponin is derived from Quillaja saponaria, such as Quillaja Saponaria bark, such as QS-21 (Quillaja saponaria Molina, fraction 21), optionally wherein the MPL is 3-O-desacyl-4′-monophosphoryl lipid A.

28. The composition of claim 21, further comprising a pharmaceutically acceptable excipient.

29. The composition of claim 21, which is in lyophilized form.

30. The composition of claim 21, which is in aqueous form.

31. The composition of claim 21, which is in unit dosage form.

32. The composition of claim 21, which, when administered in an effective amount to a mammalian subject, elicits an immune response to HIV, optionally wherein the immune response is at least a partially protective immune response.

33. The composition of claim 21 for use in raising an immune response to HIV in a mammalian subject, optionally wherein the mammalian subject is a human, more particularly wherein the human is an adult.

34. The composition of claim 21 for use in medicine.

35. The composition of claim 21 for use in the treatment or prevention of HIV.

36. A method of raising an immune response to HIV in a mammalian subject comprising the step of administering an effective amount of the composition of any one of the preceding claims to the subject, wherein the mammalian subject is a human, more particularly wherein the human is an adult or adolescent, such as an adolescent prior to sexual debut, a pediatric subject, or neonate, such as a neonate born of an HIV+ mother.

37. A method of vaccinating a mammalian subject using the composition of claim 21.

38. Use of the composition of claim 21 in the manufacture of a medicament for raising an immune response to HIV in a mammalian subject, optionally wherein the mammalian subject is a human, more particularly wherein the human is an adult.

39. The use of claim 38, wherein: the subject is a human that has previously been administered (or is concurrently administered or sequentially administered) a nucleic acid encoding one or more HIV antigens, optionally wherein the nucleic acid encodes HIV env, gag, pol, or a combination thereof, optionally wherein the nucleic acid is in the form of a viral vector, such as an inert canarypox vector, or an MVA or NYVAC pox vector; optionally wherein the nucleic acid is administered 1, 2, 3, 4, or more times, optionally where the nucleic acid may be administered sequentially or concurrently; and/or
the immune response is a polyfunctional antibody response (such as raising lgG1, lgG3, or lgG1 and lgG3; optionally wherein the antibodies elicit ADCC, ADCP, ADCVI, CD107a degranulation, IFN-gamma, MIP-1 beta, polyfuncitonal NK activation, virion capture, virus neutralization, C mediated effector function and combinations thereof, including 2, 3, 4, 5, 6, 7, 8, or all 9 of the foregoing), polyfunctional cellular response (such as production of CD40L, IL-2, IL-4, IFN-gamma, TNF-alpha, or in particular embodiments, a combination thereof, e.g., 2, 3, 4, or all 5 markers, or polyfunctional antibody response and polyfunctional cellular response.
